(19) United States
(12) Patent Application Publication
(10) Pub. No.: US 2014/0343477 A1
(43) Pub. Date: Nov. 20, 2014

(54) PANCREATIOBILIARY DIVERSION DEVICE

(71) Applicant: ENDOBETIX LTD, Misgav Business Park, (IL)

(72) Inventors: Pierre Sharvit, Emeq Hefer (IL); Chen Porat, Kiryat Tivon (IL); Dan Kinarty, Haifa (IL); Roni Zvuloni, Haifa (IL); Shlomo Lewkowicz, Kiryat Tivon (IL); Eyal Teichman, Hod Hasharon (IL)

(73) Assignee: Endobetix Ltd., Israel (IL)

(21) Appl. No.: 14/373,165

(22) PCT Filed: Jan. 17, 2013
(86) PCT No.: PCT/IL2013/050054

§ 371 Date: Jul. 18, 2014

Related U.S. Application Data
(60) Provisional application No. 61/588,371, filed on Jan. 19, 2012.

(51) Int. Cl. A61F 5/00

(52) U.S. Cl. CPC A61F 5/0076
USPC 604/9; 604/8

(57) ABSTRACT

Apparatus (200) for use with pancreaticobiliary secretions that enter a gastrointestinal tract at an anatomical location. A secretion-diversion guide (370) comprises: a proximal portion (124); a distal portion (126); first and second anchors (36/38) coupled to the proximal and distal portions which apply pressure to the tract to maintain guide (370) in place; a collection-facilitation tube (280), between the anchors, having an inner surface (245) and an outer surface (246), the inner surface defining a lumen for passage of food. The outer surface inhibits contact of the secretions with food and defines a space (7) between the outer surface and the tract. A diversion tube (290) is in communication with the space and passes secretions from the space distally, and inhibits food-secretion contact. An intragastric anchor (170/171/172) coupled to guide (370), located in the stomach, maintains the guide in place. Other applications are also described.

### CROSS-REFERENCES TO RELATED APPLICATIONS

The present application claims the priority of U.S. Provisional Application 61/588,371 to Sharvit et al., entitled, “Pancreatiobiliary diversion device,” filed on Jan. 19, 2012, which is incorporated herein by reference.

The present application is related to:

U.S. Provisional Application 61/366,586 to Sharvit et al., entitled, “Pancreatiobiliary diversion device,” filed on Jul. 22, 2010, which is incorporated herein by reference; and

PCT Application PCT/IL2011/000579 to Sharvit et al., which published as WO/2012/011105 entitled, “Pancreatiobiliary diversion device,” filed on Jul. 20, 2011, which is incorporated herein by reference.

### FIELD OF THE APPLICATION

Embodiments of the present invention relate generally to treatment of obesity and other conditions, and particularly to treatment of obesity and other conditions by diversion of endogenous secretions.

### BACKGROUND OF THE APPLICATION

The human gastrointestinal tract is a system by which ingested food is digested and absorbed in order to provide the body with essential nutrients. The human gastrointestinal tract includes the small intestine, which is the longest portion of the digestive tract. The small intestine has three sections: the duodenum, jejunum and ileum. The duodenum, where most chemical digestion takes place, precedes the jejunum and ileum and is the shortest part (typically 25-30 cm in length) of the small intestine. The duodenum begins with the duodenal bulb and ends at the ligament of Treitz.

The digestion process is regulated by several hormones, some of which are released by the gastrointestinal tract. Additionally, many digestive enzymes are secreted by the gastrointestinal tract and the pancreas to aid in the digestion of food. Other endogenous secretions, such as bile, facilitate the digestion of lipids in the small intestine. Bile is typically stored in the gallbladder and upon eating is discharged into the duodenum.

Obesity and type II diabetes are serious health concerns. It is believed that obesity promotes insulin resistance, and has been found to play an important role in the pathogenesis of diabetes. Accordingly, weight loss is generally recommended, in order to lower elevated blood glucose levels in overweight and obese individuals with type II diabetes.

Some weight loss surgical techniques currently include several types of bariatric surgical procedures, including malabsorptive procedures, e.g., biliopancreatic diversion and biliopancreatic diversion with a duodenal switch. Generally, these diversion procedures, although they also reduce stomach size, are based mainly on creating malabsorption by bypassing digestion in the duodenum and other parts of the small intestine.

### SUMMARY OF APPLICATIONS

In some applications of the present invention, methods and apparatus are provided for diversion of pancreaticobiliary secretions from an anatomical entry location into a gastrointestinal tract to a location in the gastrointestinal tract that is distal to the anatomical entry location. Typically, bile and pancreatic secretions (i.e., pancreaticobiliary secretions) pass through the common bile duct and enter the duodenum at the major duodenal papilla. Some applications of the present invention comprise transferring the pancreaticobiliary secretions from the entry location e.g., at the duodenal papilla, to a location which is beyond the duodenum, e.g., beyond the ligament of Treitz which is the final section of the duodenum. In some applications, diversion of the pancreaticobiliary secretions substantially inhibits contact, in some sections of the small intestine, of the pancreaticobiliary secretions with ingested food passing within the gastrointestinal tract. Typically, inhibiting association of the pancreaticobiliary secretions with ingested food reduces emulsification and formation of micelles of ingested fat, and disrupts the process of fat digestion in the body.

For some applications, a pancreaticobiliary secretion-diversion guide that is configured for deployment within the gastrointestinal tract is provided. Typically, the pancreaticobiliary secretion-diversion guide is configured to collect the pancreaticobiliary secretions from the anatomical entry location, e.g., duodenal papilla, and deliver the pancreaticobiliary secretions to a location in the gastrointestinal tract that is distal to the duodenal papilla, e.g., beyond the duodenum (or at a more distal site within the duodenum).

In some applications, the pancreaticobiliary secretion-diversion guide has a proximal portion and a distal portion. In this context, in the specification and in the claims, “proximal” means closer to the orifice through which the guide is originally placed into the body, and “distal” means further from this orifice. For some applications, the proximal portion has a cross section that is larger than the cross section of the distal portion, in order to enable collecting of the pancreaticobiliary secretion into the guide lumen when the guide is positioned inside the gastrointestinal tract of a subject. Typically, the diversion guide is positioned in the gastrointestinal tract such that a section of the proximal portion of the guide is configured to collect at least 75% of the pancreaticobiliary secretions entering the duodenum, substantially without the diversion guide entering the duct through which the secretions pass, e.g., the common bile duct.

In some applications, the pancreaticobiliary secretion-diversion guide is shaped to define at least a portion of a tube comprising a tube wall having an inner surface and an outer surfaces, the tube wall inner surface defining a lumen for passage of partly-digested food (i.e., chyme) therethrough. Typically, the tube wall comprises an aperture portion. The aperture portion is shaped to define one or more apertures and contacts the wall of the gastrointestinal tract of the subject in order to provide contact between ingested food within the lumen and the wall of the gastrointestinal tract. The tube wall additionally comprises a channel portion, which collects pancreaticobiliary secretions from the anatomical entry location in the gastrointestinal tract, and inhibits contact of the pancreaticobiliary secretions with the food within the lumen.

Alternatively or additionally, an anchoring system is coupled to the pancreaticobiliary secretion-diversion guide, and is configured to maintain the pancreaticobiliary secretion-diversion guide in place within the gastrointestinal tract by generally applying pressure to a wall of the gastrointestinal tract. In some applications, the anchoring system comprises (a) one or more helical and/or ring anchors disposed upstream of the entry location of pancreaticobiliary secretions into the gastrointestinal tract (e.g., duodenal papilla) and (b) one or more helical and/or ring anchors disposed downstream of the entry location of the pancreaticobiliary secretions. Each of the above anchors is typically disposed entirely within the lumen of the gastrointestinal tract, and does not penetrate tissue of the gastrointestinal tract. For example, in order to inhibit penetration of tissue, the anchors may have rounded tips. The upstream anchors may be disposed within the stomach or within the duodenal bulb.

Particular applications of the pancreaticobiliary secretion-diversion guide and of the anchoring system are described herein.

There is therefore provided, in accordance with an application of the present invention, apparatus for use with pancreaticobiliary secretions that enter a gastrointestinal tract of a subject at an anatomical entry location, the apparatus including:

a pancreaticobiliary secretion-diversion guide configured to collect the pancreaticobiliary secretions from the anatomical entry location and deliver the pancreaticobiliary secretions to a location in the gastrointestinal tract that is distal to the anatomical entry location; and

an anchoring system including one or more helical anchors located within the gastrointestinal tract and configured to apply pressure to a wall of the gastrointestinal tract in order to maintain the pancreaticobiliary secretion-diversion guide in place.

For some applications, the anatomical entry location includes a duodenal papilla of the subject, and the pancreaticobiliary secretion-diversion guide is configured to collect the pancreaticobiliary secretions that are secreted from the duodenal papilla.

For some applications, the pancreaticobiliary secretion-diversion guide is configured to collect the pancreaticobiliary secretions that are secreted from the duodenal papilla without entering a duct through which the secretions pass.

For some applications, the pancreaticobiliary secretion-diversion guide is configured to deliver the pancreaticobiliary secretions to a location that is beyond a ligament of Treitz of the subject.

For some applications, the pancreaticobiliary secretion-diversion guide is configured to deliver the pancreaticobiliary secretions to a location in the gastrointestinal tract that is 40-80 cm beyond the ligament of Treitz.

For some applications, the pancreaticobiliary secretion-diversion guide is configured to prevent the pancreaticobiliary secretions from contacting chyme in some portions of the gastrointestinal tract.

For some applications, the pancreaticobiliary secretion-diversion guide includes a proximal portion thereof and a distal portion thereof, the proximal portion having an area for collecting the secretions that is large enough to cover a five diameter hole.

For some applications, the pancreaticobiliary secretion-diversion guide includes a proximal end at a proximal portion thereof and a distal end at a distal portion thereof, the proximal portion having a cross section of 10-100 mm2, 10 mm from the proximal end, the distal portion having a cross section of 5-100 mm2, 10 mm from the distal end, the cross section of the proximal portion being at least two times greater than the cross section of the distal portion.

For some applications, the proximal portion has a length of 1-6 cm extending from the proximal end to a distal end thereof.

For some applications, the proximal portion has a width of 5-25 mm.

For some applications, the proximal portion has a width of 10-15 mm.

For some applications, the distal portion has a length of 50-110 cm extending from a proximal end thereof to the distal end thereof.

For some applications, the proximal portion is configured to collect at least 75% of the pancreaticobiliary secretions that are secreted into the gastrointestinal tract.

For some applications, the one or more helical anchors are configured to be disposed entirely within the lumen of the gastrointestinal tract, and not to penetrate tissue of the gastrointestinal tract.

For some applications, the anchoring system includes tissue-penetrating anchors configured to penetrate tissue of the gastrointestinal tract in order to maintain the pancreaticobiliary secretion-diversion guide in place.

For some applications, the one or more helical anchors are flexible.

For some applications, the one or more helical anchors each have a diameter of 20-35 mm when unconstrained.

For some applications, the one or more helical anchors are configured to be located upstream of the anatomical entry location of the pancreaticobiliary secretions.

For some applications, the one or more helical anchors are configured to be located within a duodenal bulb of the subject.

For some applications, the one or more helical anchors each have a diameter of 30-60 mm when located within the duodenal bulb.

For some applications, the one or more helical anchors each have a length of 2-40 mm, when unconstrained, measured along a longitudinal axis of each helical anchor.

For some applications, the one or more helical anchors each have a length of 5-15 mm, when unconstrained, measured along a longitudinal axis of each helical anchor.

For some applications, the one or more helical anchors are configured to be located downstream of the anatomical entry location of the pancreaticobiliary secretions.

For some applications, the pancreaticobiliary secretion-diversion guide is arranged to provide an entry point of the pancreaticobiliary secretions into the pancreaticobiliary secretion-diversion guide that is disposed between one of the one or more helical anchors and another one of the one or more helical anchors.

For some applications, the apparatus includes one or more ring anchors located within the gastrointestinal tract and configured to apply pressure to the wall of the gastrointestinal tract in order to maintain the pancreaticobiliary secretion-diversion guide in place.

For some applications, a silicone sheath is configured to surround at least a portion of the anchoring system and to apply pressure to the wall of the gastrointestinal tract in order to maintain the pancreaticobiliary secretion-diversion guide in place.

For some applications, the one or more ring anchors are configured to be disposed entirely within the lumen of the gastrointestinal tract, and not to penetrate tissue of the gastrointestinal tract.

For some applications, the ring anchor is configured to be located downstream of the anatomical entry location of the pancreaticobiliary secretions.

For some applications, the one or more ring anchors are flexible.

For some applications, the one or more ring anchors each have a diameter of 20-35 mm.

For some applications, the one or more ring anchors are configured to be positioned at a non-perpendicular angle with respect to a longitudinal axis of the pancreaticobiliary secretion-diversion guide.

For some applications, the apparatus includes an intragastric anchor configured to be located in a pyloric antrum of a stomach of the subject and configured to apply pressure to a wall of the stomach in order to maintain the pancreaticobiliary secretion-diversion guide in place.

For some applications, the intragastric anchor includes a helical anchor.

For some applications, the intragastric anchor has a longest dimension of 35-55 mm.

For some applications, the apparatus includes a retrieval element fixedly coupled to the anchoring system, configured for facilitating endoscopic retrieval of the apparatus.

For some applications, the apparatus includes at least one anchoring mount configured to be located in the gastrointestinal tract, downstream of the one or more helical anchors, the anchoring mount including two or more longitudinal struts.

For some applications, the anchoring mount is configured to be disposed entirely within the lumen of the gastrointestinal tract, and not to penetrate tissue of the gastrointestinal tract.

For some applications, at least one of the two or more longitudinal struts is configured to be aligned in parallel with a longitudinal axis of the gastrointestinal tract and at least one of the two or more longitudinal struts is a curved strut which is configured to contact the wall of the gastrointestinal tract to apply pressure thereto.

There is further provided, in accordance with an application of the present invention, apparatus for use with pancreaticobiliary secretions that enter a gastrointestinal tract of a subject at an anatomical entry location, the apparatus including:

a pancreaticobiliary secretion-diversion guide shaped to define a pancreaticobiliary secretion-diversion guide lumen having a proximal end at a proximal portion thereof and a distal end at a distal portion thereof, the proximal portion having a cross section of 10-100 mm2, 10 mm from the proximal end, the distal portion having a cross section of 5-100 mm2, 10 mm from the distal end, the cross section of the proximal portion being at least two times greater than the cross section of the distal portion; and

an anchoring system configured to be located within the gastrointestinal tract and to apply pressure to a wall of the gastrointestinal tract in order to maintain the pancreaticobiliary secretion-diversion guide in place,

the proximal portion of the pancreaticobiliary secretion-diversion guide is configured to collect pancreaticobiliary secretions from the anatomical entry location and the distal end of the pancreaticobiliary secretion-diversion guide is configured to deliver the pancreaticobiliary secretions to a location in the gastrointestinal tract that is distal to the anatomical entry location.

For some applications, the proximal portion is configured to collect at least 75% of the pancreaticobiliary secretions that are secreted into the gastrointestinal tract.

For some applications, the proximal portion has a length of 1-6 cm extending from the proximal end to a distal end thereof.

For some applications, the proximal portion has a width of 5-25 mm.

For some applications, the proximal portion has a width of 10-15 mm.

For some applications, the distal portion has a length of 50-110 cm extending from a proximal end thereof to the distal end thereof.

For some applications, the distal end is configured to deliver the pancreaticobiliary secretions to a location in the gastrointestinal tract that is 40-80 cm beyond a ligament of Treitz of the subject.

There is still further provided, in accordance with an application of the present invention, apparatus for use with pancreaticobiliary secretions that enter a gastrointestinal tract of a subject at an anatomical entry location, the apparatus including:

a pancreaticobiliary secretion-diversion guide configured to collect pancreaticobiliary secretions from the anatomical entry location and deliver the pancreaticobiliary secretions to a location in the gastrointestinal tract that is distal to the anatomical entry location; and

an anchoring system configured to apply pressure to a wall of the gastrointestinal tract in order to maintain the pancreaticobiliary secretion-diversion guide in place, the anchoring system including:

one or more helical anchors configured to be disposed upstream of the entry location of pancreaticobiliary secretions into the gastrointestinal tract; and

one or more ring anchors configured to be disposed downstream of the entry location of the pancreaticobiliary secretions.

For some applications, the one or more helical anchors each have a diameter of 20-35 mm when unconstrained.

For some applications, the one or more helical anchors are configured to be located within a duodenal bulb of the subject.

For some applications, the one or more helical anchors each have a diameter of 30-60 mm when located within the duodenal bulb.

For some applications, the one or more helical anchors each have a length of 2-40 mm, when unconstrained, measured along a longitudinal axis of each helical anchor.

For some applications, the one or more helical anchors each have a length of 5-15 mm, when unconstrained, measured along a longitudinal axis of each helical anchor.

For some applications, the one or more helical anchors are flexible.

For some applications, the one or more ring anchors are flexible.

For some applications, the one or more ring anchors each have a diameter of 20-35 mm when unconstrained.

For some applications, the one or more ring anchors are configured to be positioned at a non-perpendicular angle with respect to a longitudinal axis of the pancreaticobiliary secretion-diversion guide.

For some applications, the apparatus includes an intragastric anchor configured to be located in a lumen of a pyloric antrum of a stomach of the subject and configured to apply pressure to a wall of the stomach in order to maintain the pancreaticobiliary secretion-diversion guide in place.

For some applications, the intragastric anchor includes a helical anchor.

For some applications, the pancreaticobiliary secretion-diversion guide is arranged to provide an entry point of the pancreaticobiliary secretions into the pancreaticobiliary secretion-diversion guide which is disposed between one of the one or more helical anchors and one of the one or more ring anchors.

For some applications, the one or more helical anchors and the one or more ring anchors are configured to be disposed entirely within the lumen of the gastrointestinal tract, and not to penetrate tissue of the gastrointestinal tract.

There is additionally provided, in accordance with an application of the present invention, apparatus for use with pancreaticobiliary secretions that enter a gastrointestinal tract of a subject at an anatomical entry location, the apparatus including:

a pancreaticobiliary secretion-diversion guide configured to collect pancreaticobiliary secretions from the anatomical entry and deliver the pancreaticobiliary secretions to a location in the gastrointestinal tract that is distal to the anatomical entry location; and

an anchoring system configured to apply pressure to a wall of the gastrointestinal tract in order to maintain the pancreaticobiliary secretion-diversion guide in place, the anchoring system including:

one or more helical anchors configured to be disposed upstream of the entry location of pancreaticobiliary secretions into the gastrointestinal tract; and

one or more helical anchors configured to be disposed downstream of the entry location of pancreaticobiliary secretions into the gastrointestinal tract.

For some applications, the one or more helical anchors each have a diameter of 20-35 mm when unconstrained.

For some applications, the one or more helical anchors configured to be disposed upstream of the entry location of pancreaticobiliary secretions into the gastrointestinal tract, are configured to be located within a duodenal bulb of the subject.

For some applications, the one or more helical anchors each have a diameter of 30-60 mm when located within the duodenal bulb.

For some applications, the one or more helical anchors each have a length of 2-40 mm, when unconstrained, measured along a longitudinal axis of each helical anchor.

For some applications, the one or more helical anchors each have a length of 5-15 mm, when unconstrained, measured along a longitudinal axis of each helical anchor.

For some applications, the one or more helical anchors are flexible.

For some applications, the one or more helical anchors are configured to be positioned at a non-perpendicular angle with respect to a longitudinal axis of the pancreaticobiliary secretion-diversion guide.

For some applications, the apparatus includes an intragastric anchor configured to be located in a pyloric antrum of a stomach of the subject and configured to apply pressure to a wall of the stomach in order to maintain the pancreaticobiliary secretion-diversion guide in place.

For some applications, the intragastric anchor includes a helical anchor.

For some applications, the pancreaticobiliary secretion-diversion guide is arranged to provide an entry point of the pancreaticobiliary secretions into the pancreaticobiliary secretion-diversion guide that is disposed between one of the one or more helical anchors configured to be disposed upstream of the entry location of the pancreaticobiliary secretions, and one of the one or more helical anchors configured to be disposed downstream of the entry location of the pancreaticobiliary secretions.

For some applications, the one or more helical anchors are configured to be disposed entirely within the lumen of the gastrointestinal tract, and not to penetrate tissue of the gastrointestinal tract.

There is yet additionally provided, in accordance with an application of the present invention, apparatus for use with pancreaticobiliary secretions that enter a gastrointestinal tract of a subject at an anatomical entry location, the apparatus including:

a pancreaticobiliary secretion-diversion guide configured to collect pancreaticobiliary secretions from the anatomical entry location and deliver the pancreaticobiliary secretions to a location in the gastrointestinal tract that is distal to the anatomical entry location;

an anchoring system configured to apply pressure to a wall of the gastrointestinal tract in order to maintain the pancreaticobiliary secretion-diversion guide in place including:

one or more helical anchors configured to be located upstream of the entry location of pancreaticobiliary secretions into the gastrointestinal tract; and

at least one anchoring mount configured to be located in the gastrointestinal tract, downstream of the one or more helical anchors, the anchoring mount including two or more longitudinal struts, configured to be aligned in parallel with a longitudinal axis of the gastrointestinal tract.

For some applications, the one or more helical anchors and the at least one anchoring mount are configured to be disposed entirely within the lumen of the gastrointestinal tract, and not to penetrate tissue of the gastrointestinal tract.

There is still additionally provided, in accordance with an application of the present invention, apparatus for use with pancreaticobiliary secretions that enter a gastrointestinal tract of a subject at an anatomical entry location, the apparatus including:

a pancreaticobiliary secretion-diversion guide shaped to define at least a portion of a tube, the tube including a tube wall, the tube wall having an inner surface and an outer surface, the tube wall inner surface defining a lumen for passage of food therethrough,

the tube wall includes:

an aperture portion, shaped to define one or more apertures and configured to contact a wall of a gastrointestinal tract of a subject and to provide contact between food within the lumen and the wall of the gastrointestinal tract; and

a channel portion, configured to collect pancreaticobiliary secretions from the anatomical entry location, and to inhibit contact of the pancreaticobiliary secretions with the food within the lumen.

For some applications, the one or more apertures include between 1-5 apertures.

For some applications, the one or more apertures include between 5-50 apertures.

For some applications, each of the one or more apertures has a cross sectional area of at least 1 cm2.

For some applications, the one or more apertures are configured to provide contact between chyme within the lumen of the tube wall inner surface and the wall of the gastrointestinal tract.

For some applications, the pancreaticobiliary secretion-diversion guide has a length of 60-110 cm.

For some applications, the tube wall inner surface lumen has a diameter of 20-35 mm.

There is further additionally provided, in accordance with an application of the present invention, apparatus for use with pancreaticobiliary secretions that enter a gastrointestinal tract of a subject at an anatomical entry location, the apparatus including:

a pancreaticobiliary secretion-diversion guide configured to transfer pancreaticobiliary secretions from the anatomical entry location to a location in the gastrointestinal tract that is distal to the anatomical entry location; and

one or more arms, coupled to the guide, and configured to apply pressure to the wall of the gastrointestinal tract in order to maintain the pancreaticobiliary secretion-diversion guide in place in the gastrointestinal tract.

For some applications, the pancreaticobiliary secretion-diversion guide is shaped to define a channel in fluid communication with the wall of the gastrointestinal tract when the guide is in the gastrointestinal tract.

For some applications, the pancreaticobiliary secretion-diversion guide is configured to inhibit contact in some sections of the gastrointestinal tract between the pancreaticobiliary secretions and chyme passing through the gastrointestinal tract.

For some applications, the pancreaticobiliary secretion-diversion guide has a length of 60-110 cm.

For some applications, the pancreaticobiliary secretion-diversion guide is configured to deliver the pancreaticobiliary secretions to a location in the gastrointestinal tract that is 40-80 cm beyond the ligament of Treitz.

There is yet additionally provided, in accordance with an application of the present invention, apparatus for use with pancreaticobiliary secretions that enter a gastrointestinal tract of a subject at an anatomical entry location, the apparatus including:

a pancreaticobiliary secretion-diversion guide shaped to define at least a portion of a tube, the tube including a tube wall, the tube wall having an inner surface and an outer surface, the tube wall inner surface defining a lumen for passage of food therethrough,

the tube wall includes:

an aperture portion, shaped to define one or more apertures and configured to contact a wall of a gastrointestinal tract of a subject and to provide contact via the apertures between food within the lumen and the wall of the gastrointestinal tract; and

an inner tube shaped to define a lumen coupled to the inner surface of the tube wall, configured to transfer pancreaticobiliary secretions from the anatomical entry location to a location in the gastrointestinal tract that is distal to the anatomical entry location.

For some applications, the lumen of the tube wall inner surface has a diameter of 20-35 mm.

For some applications, the pancreaticobiliary secretion-diversion guide has a length of 60-110 cm.

For some applications, the one or more apertures include between 1-5 apertures.

For some applications, the one or more apertures include between 5-50 apertures.

For some applications, each of the one or more apertures has a cross sectional area of at least 1 cm2.

For some applications, the inner tube is shaped to define an orifice, in a lateral wall of the lumen, having a diameter of 5-20 mm.

For some applications, the inner tube has a length of 60-110 cm.

There is yet further provided, in accordance with an application of the present invention, a method, including:

deploying within a gastrointestinal tract a pancreaticobiliary secretion-diversion guide configured to collect pancreaticobiliary secretions from an anatomical entry location and deliver the pancreaticobiliary secretions to a location in the gastrointestinal tract that is distal to the anatomical entry location; and

implanting an intragastric anchor, coupled to the guide, in a pyloric antrum of a stomach of a subject, the anchor configured to apply pressure to a wall of the stomach in order to maintain the guide in place.

For some applications, the method includes implanting in a small intestine of the subject an anchoring system coupled to the guide, the anchoring system including one or more anchors configured to apply pressure to a wall of the gastrointestinal tract in order to maintain the guide in place.

There is additionally provided in accordance with an application of the present invention, apparatus for use with pancreaticobiliary secretions that enter a gastrointestinal tract of a subject at an anatomical entry location, the apparatus including:

a pancreaticobiliary secretion-diversion guide, including:
a proximal portion;
a distal portion;
first and second anchors coupled to the proximal and distal portions respectively and configured to apply pressure to the wall of the gastrointestinal tract in order to maintain the pancreaticobiliary secretion-diversion guide in place within the gastrointestinal tract;
a collection-facilitation tube, between the first and second anchors, having an inner surface and an outer surface, the inner surface defining a lumen for passage of food therethrough, the outer surface disposed to inhibit contact of the secretions with food within the lumen and configured to define a space between the outer surface and the gastrointestinal tract for collecting the secretions;
a diversion tube, configured to:
   be in fluid communication with the space between the outer surface and the gastrointestinal tract,
   pass pancreaticobiliary secretions from the space to a location in the gastrointestinal tract that is distal to the anatomical entry location, and
   inhibit contact of the pancreaticobiliary secretions with food that has passed through the lumen; and

an intragastric anchor coupled to the pancreaticobiliary secretion-diversion guide, and configured to be located in a stomach of the subject and to maintain the pancreaticobiliary secretion-diversion guide in place.

For some applications, the outer surface of the collection-facilitation tube is shaped to define one or more grooves configured to facilitate collection of the pancreaticobiliary secretions into the diversion tube.

For some applications, the diversion tube includes one or more unidirectional valves configured to direct the flow of pancreaticobiliary secretions in a distal direction in the diversion tube.

For some applications, the collection-facilitation tube is shaped to define a frustoconical collection-facilitation tube.

For some applications, the anchors include ring anchors.

For some applications, each ring anchor has a diameter of 20-35 mm.

For some applications, the intragastric anchor is toroidal and has a center line diameter of 15-100 mm.

For some applications, the intragastric anchor has a center line diameter of 15-35 mm.

For some applications, the intragastric anchor has a center line diameter of 18-25 mm.

For some applications, the intragastric anchor is toroidal and has a cross-sectional diameter of 2-10 mm.

For some applications, the apparatus further includes one or more flexible connecting elements which couple the intragastric anchor to the pancreaticobiliary secretion-diversion guide.

For some applications, the one or more flexible connecting elements couple the intragastric anchor to the first anchor.

For some applications, the flexible connecting elements are configured to deform in response to peristaltic motion of the gastrointestinal tract.

For some applications, the flexible connecting elements include one or more helically-shaped elements.

For some applications, the diversion tube has a diameter of 4-20 mm.

For some applications, the collection-facilitation tube has a diameter of 20-25 mm.

For some applications, the apparatus further includes a support anchor configured to be disposed proximally to the collection-facilitation tube and to apply pressure to the wall of the gastrointestinal tract in order to maintain the pancreaticobiliary secretion-diversion guide in place within the gastrointestinal tract.

For some applications, the support anchor is configured to be located within a duodenal bulb of the subject.

For some applications, the support anchor is toroidal and has a center line diameter of 25-40 mm when unconstrained.

For some applications, the support anchor is toroidal and has a center line diameter of 30-60 mm when deployed within the gastrointestinal tract.

For some applications, the support anchor is toroidal and has a cross-sectional diameter of 2-6 mm when unconstrained.

For some applications, the apparatus further includes one or more flexible connecting elements which couple the support anchor to the pancreaticobiliary secretion-diversion guide.

For some applications, the one or more flexible connecting elements couple the support anchor to the first anchor of the pancreaticobiliary secretion-diversion guide.

For some applications, the flexible connecting elements are configured to deform in response to peristaltic motion of the gastrointestinal tract.

For some applications, the flexible connecting elements include one or more helically-shaped elements.

For some applications, a proximal portion of the diversion tube has a cross section that is larger than a cross section of a distal portion of the diversion tube.

For some applications, the pancreaticobiliary secretion-diversion guide does not include a stent that surrounds the collection-facilitation tube.

The present invention will be more fully understood from the following detailed description of embodiments thereof, taken together with the drawings, in which:

### BRIEF DESCRIPTION OF THE DRAWINGS

FIGS. 1A-C are schematic illustrations of apparatus for diversion of pancreaticobiliary secretions, in accordance with some applications of the present invention;

FIGS. 2A-B are schematic illustrations of an alternative configuration of the anchoring system of the apparatus shown in FIGS. 1A-B, in accordance with some applications of the present invention;

FIGS. 3A-B are schematic illustrations of another alternative configuration of the anchoring system, in accordance with some applications of the present invention;

FIGS. 4A-B are schematic illustrations of an alternative configuration of the anchoring system of the apparatus shown in FIGS. 3A-B, in accordance with some applications of the present invention;

FIGS. 5A-B are schematic illustrations of an anchoring system for use with a pancreaticobiliary secretion-diversion guide, in accordance with some applications of the present invention;

FIGS. 6A-B are schematic illustrations of an alternative configuration of the anchoring system for use with a pancreaticobiliary secretion-diversion guide as shown in FIGS. 5A-B, in accordance with an application of the present invention;

FIGS. 7A-B are schematic illustrations of an anchoring system for use with a pancreaticobiliary secretion-diversion guide, in accordance with an application of the present invention;

FIGS. 8A-B are schematic illustrations of a pancreaticobiliary secretion-diversion guide, in accordance with an application of the present invention;

FIGS. 9A-B are schematic illustrations of a pancreaticobiliary secretion-diversion guide, in accordance with an application of the present invention;

FIGS. 10A-B are schematic illustrations of a pancreaticobiliary secretion-diversion guide, in accordance with an application of the present invention;

FIGS. 11A-B are schematic illustrations of apparatus for use with pancreaticobiliary secretions, in accordance with an application of the present invention;

FIGS. 12A-B are schematic illustrations of apparatus for use with pancreaticobiliary secretions, in accordance with an application of the present invention;

FIGS. 13A-B are schematic illustrations of apparatus for use with pancreaticobiliary secretions, in accordance with an application of the present invention;

FIGS. 14A-B are schematic illustrations of a pancreaticobiliary secretion-diversion guide, in accordance with an application of the present invention;

FIGS. 15A-B are schematic illustrations of a pancreaticobiliary secretion-diversion guide, in accordance with an application of the present invention; and

FIGS. 16A-B are schematic illustrations of a diversion tube of a pancreaticobiliary secretion-diversion guide, in accordance with an application of the present invention.

### DETAILED DESCRIPTION OF APPLICATIONS

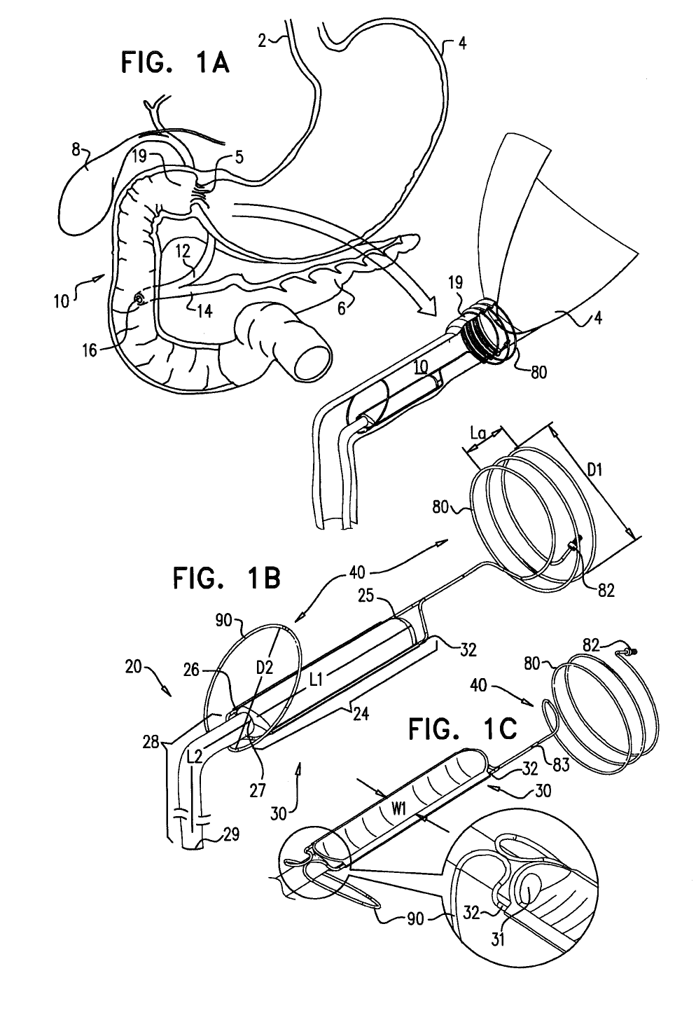

Reference is made to FIGS. 1A-B, which are schematic illustrations of apparatus 20 for diversion of pancreaticobiliary secretions, in accordance with some applications of the present invention. Apparatus 20 is typically configured for placement inside a gastrointestinal tract of a subject. FIG. 1A provides a schematic illustration of several components of a human digestive system. During the process of food digestion, food passes through esophagus 2 into stomach 4. The content of stomach 4 passes through pylorus 5 into the first section of the small intestine, duodenum 10. Bile, which aids in the process of fat digestion, is stored between meals in gallbladder 8. When the bile is released from gallbladder 8, it flows through the cystic duct and the common bile duct 12. Pancreas 6 produces exocrine secretions, including digestive enzymes, which pass through pancreatic duct 14. Pancreatic duct 14 merges with common bile duct 12 and together they form, at a medial side of a second portion of duodenum 10, a structure called the major duodenal papilla 16. Thus, major duodenal papilla 16, is an anatomical entry location of pancreaticobiliary secretions into the gastrointestinal tract. In some cases, common bile duct 12 discharges into the duodenum through a papilla which is in close proximity to major duodenal papilla 16. It will be appreciated that some applications of the present are applicable to such cases.

As shown in FIGS. 1A-C, some applications of the present invention comprise apparatus 20 comprising a pancreaticobiliary secretion-diversion guide 30 and anchoring system 40. Guide 30 is configured to collect pancreaticobiliary secretions from an anatomical entry location into duodenum 10, e.g., duodenal papilla 16, and deliver the pancreaticobiliary secretions to a location in the gastrointestinal tract that is distal to the duodenal papilla. For some applications, guide 30 transfers the pancreaticobiliary secretions to a location that is beyond the duodenum, e.g., beyond the ligament of Treitz, which is the final section of the duodenum. For some applications, guide 30 diverts the pancreaticobiliary secretions to a location that is at least 40 cm or less than 80 cm or between 40 and 80 cm beyond the ligament of Treitz. For some applications, guide 30 is configured to divert the pancreaticobiliary secretions to any location within the small intestine that is distal to the ligament of Treitz.

Guide 30 is inserted into a gastrointestinal tract of a subject. Guide 30 is typically disposed within duodenum 10 in a location that is in the vicinity of duodenal papilla 16, such that secretions entering the duodenum at papilla 16 are directly collected into guide 30. The pancreaticobiliary secretions flow through guide 30 and are typically discharged from the guide in an area that is in the upper and/or mid jejunum. It is to be noted that for some applications guide 30 is configured (i.e., sufficient in length) to deliver the pancreaticobiliary secretions to a location that is in the lower jejunum or the ileum of the small intestine.

Guide 30 generally prevents the pancreaticobiliary secretions from contacting at least a portion of the wall of the duodenum. Additionally, guide 30 is typically impermeable, thus isolating the pancreaticobiliary secretions and substantially inhibiting contact, in some sections of the small intestine, between the pancreaticobiliary secretions and partly digested food passing within the gastrointestinal tract (generally referred to as “chyme”). Typically, inhibiting contact of the pancreaticobiliary secretions with chyme reduces emulsification and formation of micelles of ingested fat, and disrupts the process of fat digestion in the body.

Reference is made to FIG. 1B. Typically, pancreaticobiliary secretion-diversion guide 30 has a proximal portion 24 comprising a proximal end 25 and a distal end 26, and a distal portion 28, which is shaped to define a pancreaticobiliary secretion-diversion guide lumen, comprising a proximal end 27 and a distal end 29. Proximal portion 24 typically has a length L1 of at least 1 cm or less than 6 cm or between 1 cm and 6 cm that extends from proximal end 25 to distal end 26. Proximal portion 24 typically has a width W of at least 5 mm (e.g., at least 10 mm) or less than 25 mm (e.g., less than 15 mm) or between 10 and 15 mm. Distal portion 28 typically has a length L2 of at least 50 cm or less than 110 cm or between 50 and 110 cm that extends from proximal end 27 to distal end 29. It is to be noted that proximal portion 24 is shown as having a generally semi-elliptical cross-section by way of illustration and not limitation. Proximal portion 24 may have any suitable shape, e.g., semi-circular.

FIGS. 1A-C show proximal portion 24 and distal portion 28 of guide 30 as discrete segments by way of illustration and not limitation. For other applications guide 30 may comprise a single component, e.g., a single hollow cylinder tube. Alternatively, proximal portion 24 and distal portion 28 of guide 30 are discrete segments having a smooth transition along the length of the tube.

Typically, as shown in FIG. 1C, proximal portion 24 has an open, generally, concave side, in order to enable collecting of the pancreaticobiliary secretions into the guide lumen when the guide is positioned inside the gastrointestinal tract of a subject. The open side of proximal portion 24 is typically configured to contact the wall of duodenum 10 in the vicinity of papilla 16 in order to collect pancreaticobiliary secretions into guide 30. Pancreaticobiliary secretions collected by proximal portion 24 flow to distal portion 28 through orifice 31, as shown in FIG. 1C. Distal portion 28 is open at distal end 29 thereof, allowing discharge of the pancreaticobiliary secretions.

For some applications, proximal portion 24 has a cross-sectional area of 10-100 mm2, 10 mm from proximal end 25. Distal portion 28 typically has a cross-sectional area of 5-100 mm2, 10 mm from distal end 29, the cross-sectional area of proximal portion 24 typically being at least two times greater than the cross-section area of the distal portion 28. Guide 30 is positioned in the gastrointestinal tract such that proximal portion 24 of guide 30 is configured to collect at least 75% of the pancreaticobiliary secretion entering the duodenum, substantially without guide 30 entering papilla 16 and/or a duct through which the secretions pass, e.g., common bile duct 12. Additionally, proximal portion 24 may enable collection of pancreaticobiliary secretions from additional entry points of pancreaticobiliary secretions into the duodenum in the vicinity of major duodenal papilla 16. The area of proximal portion 24 that is open to the duodenal papilla, in order to receive the secretions, is typically large enough to completely cover the papilla, e.g., large enough to cover a hole (the papilla) which has a diameter of 5 mm. As shown in FIG. 1C, the opening is considerably larger than this minimum size, and is typically at least 1 cm2, e.g., at least 2 cm2, and/or is less than 20 cm2, e.g., less than 12 cm2.

For other applications, guide 30 is shaped to define a tube having a tube wall configured for passage of pancreaticobiliary secretions therethrough. Typically, at least a portion of the tube contacts the wall of duodenum 10 in the vicinity of entry locations of pancreaticobiliary secretions into duodenum 10, e.g., papilla 16. For some applications, the tube is shaped to define a wide, funnel, proximal portion configured to contact the wall of duodenum 10 in the vicinity of papilla 16 in order to collect pancreaticobiliary secretions into the tube. For such applications, the tube may become progressively smaller in cross-section towards a distal portion thereof. For such applications, the diameter of the proximal portion is at least 5 mm or less than 20 mm or between 5-20 mm, and is configured to surround at least the entire diameter of the duodenal papilla.

For some applications (not shown), the portion of guide 30 which comes in contact with the wall of duodenum 10 in the vicinity of papilla 16 is shaped to define one or more, e.g., 1-10 or 10-100 or 100-1000, apertures which allow pancreaticobiliary secretions to enter guide 30. For some applications, the apertures are 2-5 mm in diameter.

Apparatus 20 comprises anchoring system 40, which is coupled to pancreaticobiliary secretion-diversion guide 30 and functions to maintain guide 30 in place within the intestine, even while the intestine undergoes peristalsis. For some applications, anchoring system 40 comprise one or more anchors located within the gastrointestinal tract and configured to apply pressure to a wall of the gastrointestinal tract in order to maintain pancreaticobiliary secretion-diversion guide 30 in place.

For some applications, anchoring system 40 comprises one or more helical anchors disposed upstream of the entry location of pancreaticobiliary secretions into the gastrointestinal tract and one or more ring anchors disposed downstream of the entry location of the pancreaticobiliary secretions. As shown in FIGS. 1A-C, anchoring system 40 comprises helical anchor 80, which is disposed upstream of the entry location of pancreaticobiliary secretions into the gastrointestinal tract, i.e., upstream of duodenal papilla 16. Anchor 80 is shown as comprising three helical turns by way of illustration and not limitation. Anchor 80 may comprise any suitable number of helical turns. Anchor 80 is shaped to define an anchor lumen which allows passage of chyme therethrough. Anchor 80 typically assumes a diameter D1 of 30-60 mm when deployed within the gastrointestinal tract. A resting diameter D1 of anchor 80 (i.e., if not constrained by the gastrointestinal tract) is typically at least 20 mm, or less than 35 mm, or between 20-35 mm. A resting length Lₐ of anchor 80 (i.e., if not constrained by the gastrointestinal tract) is typically 2-40 mm, e.g., 5-15 mm, measured along a longitudinal axis of each helical anchor. It is to be noted that the pitch, i.e., the distance between turns of the helical anchor, may vary from one turn to the next. For some applications, anchor 80 comprises a flexible anchor. Typically, the flexibility of anchor 80 facilitates endoscopic passage of anchor 80 in a generally compressed state thereof through the esophagus.

FIG. 1A shows anchor 80 deployed within the first section of duodenum 10, duodenal bulb 19. Anchor 80 pushes against the walls of duodenal bulb 19, and applies pressure to the wall of duodenal bulb 19 in order to fix pancreaticobiliary secretion-diversion guide 30 in place. Anchor 80 typically comprises retrieval element 82 allowing safe retrieval of anchoring system 40 from within the body of the subject.

For some applications, an extension 83 from anchor 80 leads to one or more (e.g., two) longitudinal struts 32 which are coupled to proximal portion 24 of pancreaticobiliary secretion-diversion guide 30, to fix guide 30 within the intestine.

In some applications, anchoring system 40 additionally comprises one or more ring anchors 90 configured to be disposed downstream of the entry location of pancreaticobiliary secretions into the gastrointestinal tract, e.g., downstream of duodenal papilla 16. For some applications, ring anchor 90 comprises a flexible, elastic anchor, which applies an outward force to the intestine. Typically, the flexibility of anchor 90 facilitates endoscopic passage of anchor 90 through the esophagus in a highly elliptical shape. When anchor 90 is deployed within the intestine, it naturally relaxes to assume a less elliptical shape (i.e., more circular), applying a force to the intestine wall. Ring anchor 90 typically surrounds a portion of pancreaticobiliary secretion-diversion guide 30. FIGS. 1A-B show ring anchor 90 surrounding a portion of distal portion 28 of guide 30 and leaning against proximal portion 24 in order to apply pressure thereto to maintain guide 30 in place. Anchor 90 may be positioned generally perpendicularly with respect to a longitudinal axis of guide 30, or as shown in FIGS. 1A-B, at a non-perpendicular angle with respect to a longitudinal axis of guide 30. Ring anchor 90 is shaped to define an anchor lumen which allows passage of chyme therethrough. Anchor 90 typically has a diameter D2 of 20-35 mm, e.g., 25-35 mm when not constrained.

Alternatively, for some applications, anchor 80 may be disposed between the duodenal bulb and the entry location of pancreaticobiliary secretions into the gastrointestinal tract. For such applications, D1 is generally the same as D2 when deployed within the gastrointestinal tract.

For some applications, anchor 90 is configured to be disposed upstream of the entry location of pancreaticobiliary secretions into the gastrointestinal tract, e.g., upstream of duodenal papilla 16, and anchor 80 is configured to be disposed downstream of the entry location of pancreaticobiliary secretions into the gastrointestinal tract, e.g., downstream of duodenal papilla 16.

Each of the above anchors is typically disposed entirely within the lumen of the gastrointestinal tract, and does not penetrate tissue of the gastrointestinal tract. For example, in order to inhibit penetration of tissue, the anchors may have rounded tips. The upstream anchors may be disposed within the stomach or within the duodenal bulb. Alternatively, the upstream anchors may be disposed between the duodenal bulb and the entry location of pancreaticobiliary secretions into the gastrointestinal tract.

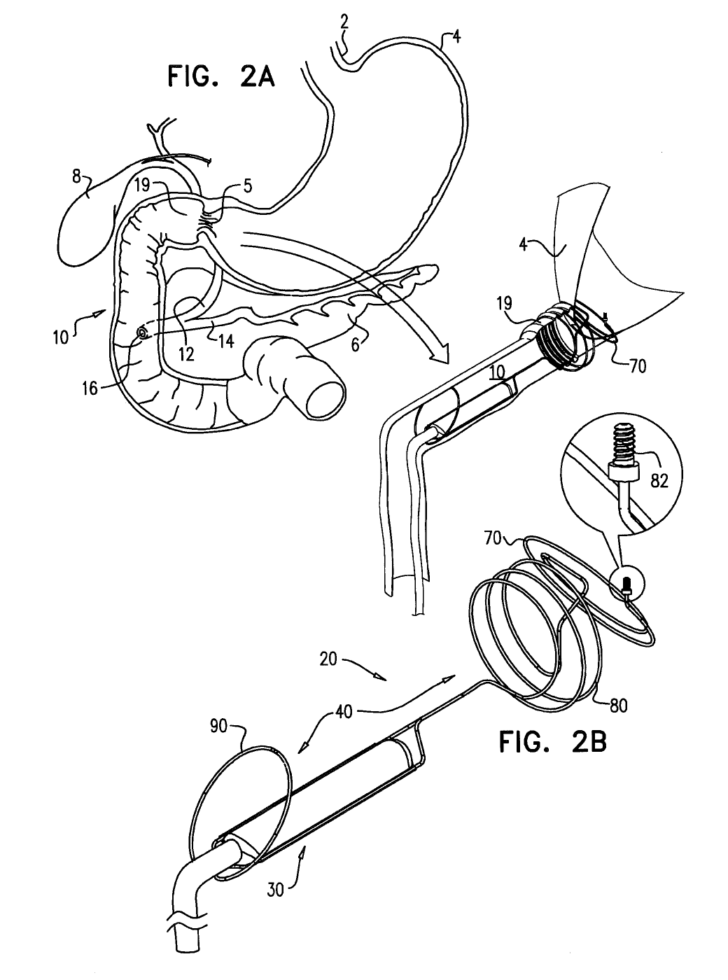

Reference is made to FIGS. 2A-B, which are schematic illustrations of an alternative configuration of the anchoring system of apparatus 20, in accordance with some applications of the present invention. For some applications, anchoring system 40 comprises an additional intragastric anchor 70, which is configured for deployment within a pyloric antrum of the stomach of the subject. Intragastric anchor 70 is coupled to helical anchor 80. Intragastric anchor 70 is shown having two helical turns by way of illustration and not limitation. Intragastric anchor 70 may comprise any suitable number of turns, or, alternatively, no turns. Typically, intragastric anchor 70 has a longest dimension of 35-55 mm. Intragastric anchor 70 is typically configured to remain within the stomach of the subject and accordingly is sufficient in size, and shaped in a manner, that prevents it from passing through the pylorus. Optionally, portions of intragastric anchor 70 contact the wall of the stomach and may apply pressure to the wall of the stomach in order to maintain guide 30 in place.

Retrieval element 82 in this application is coupled to intragastric anchor 70. An exploded view of retrieval element 82 shows element 82 shaped to define a screw in order to facilitate deployment in the gastrointestinal tract, and retrieval of apparatus 20 or portions thereof. Alternatively, element 82 may define any other suitable shape.

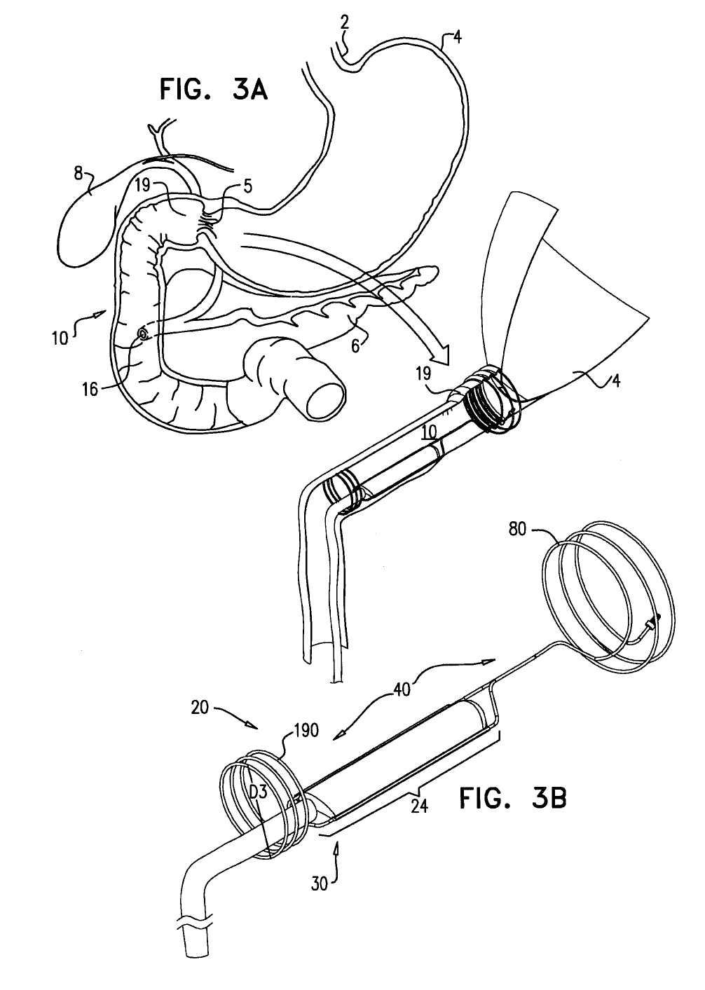

Reference is made to FIGS. 3A-B, which are schematic illustrations of another alternative configuration of the anchoring of the apparatus 20, in accordance with some applications of the present invention.

For some applications, anchoring system 40 comprises one or more helical anchors 190 which are disposed downstream of the entry location of pancreaticobiliary secretions into the gastrointestinal tract, e.g., downstream of duodenal papilla 16. Helical anchor 190 typically surrounds a portion of pancreaticobiliary secretion-diversion guide 30. FIGS. 3A-B show helical anchor 190 surrounding a portion of distal portion 28 of guide 30. Anchor 190 may be positioned generally perpendicularly with respect to a longitudinal axis of guide 30 as shown in FIGS. 3A-B, or at an angle with respect to a longitudinal axis of guide 30 (configuration not shown). Helical anchor 190 is shaped to define an anchor lumen which allows passage of chyme therethrough. Anchor 190 typically assumes a diameter D3 of 20-35 mm, e.g., 25-35 mm when unconstrained by the gastrointestinal tract. Helical anchor 190 is shown having three helical turns by way of illustration and not limitation. Anchor 190 may comprise any suitable number of helical turns. Helical anchor 190 typically contacts the wall of the intestine in order to apply pressure to the wall and maintain guide 30 in place.

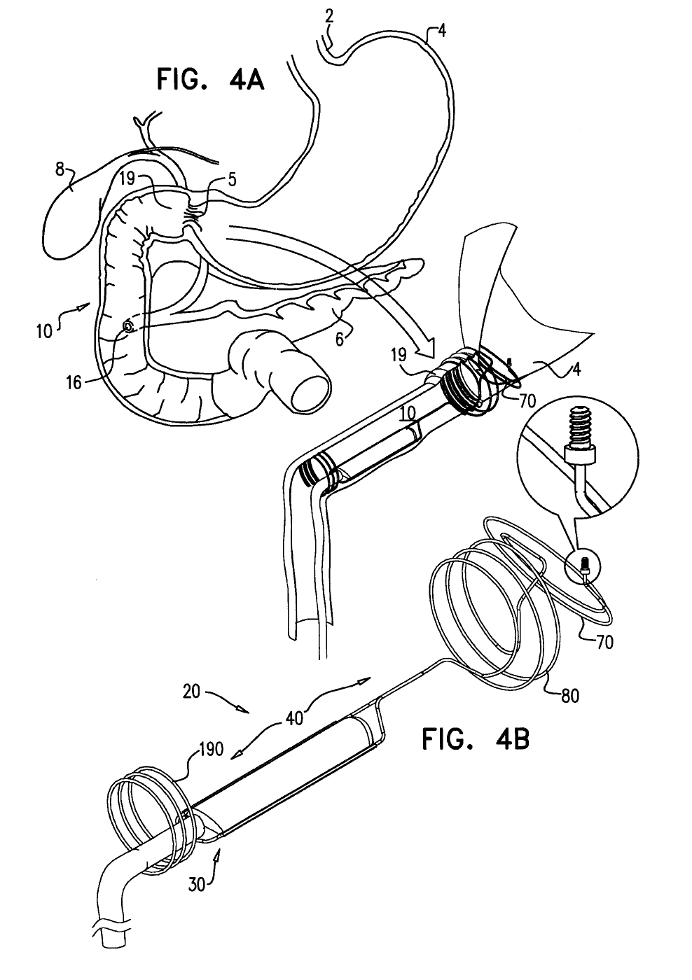

Reference is made to FIGS. 4A-B which are schematic illustrations of an alternative configuration of the anchoring system of apparatus 20, in accordance with some applications of the present invention. For some applications, anchoring system 40 comprises anchor 190 and an additional intragastric anchor 70 which is configured for deployment within the pyloric antrum of stomach 4 of the subject as described with reference to FIG. 2A-B.

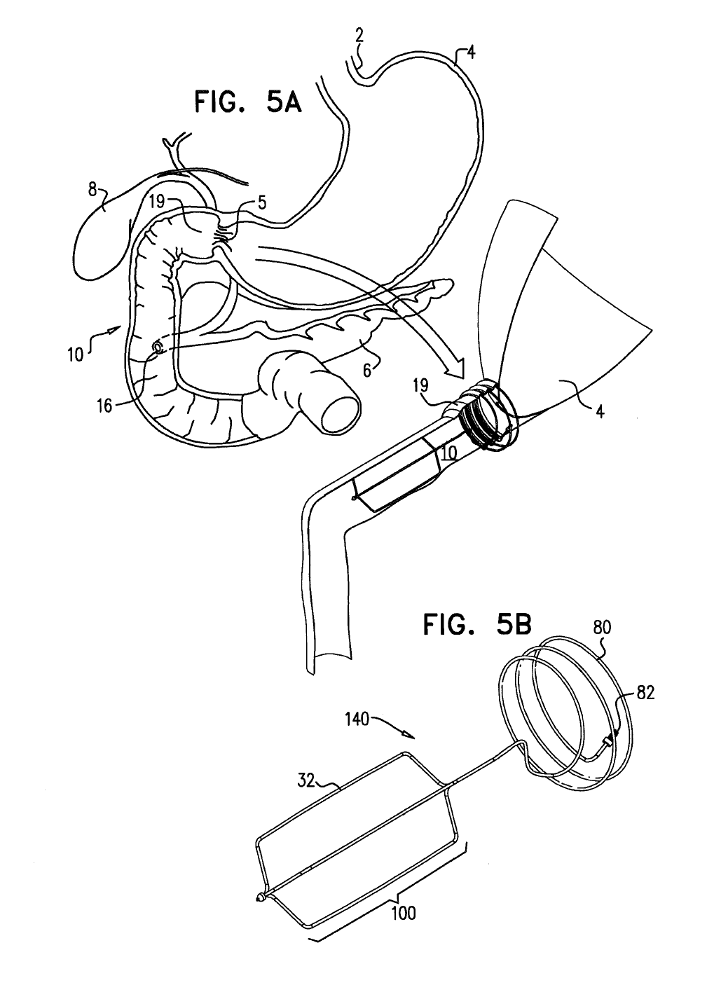

Reference is made to FIGS. 5A-B, which are schematic illustrations of anchoring system 140 for use with a pancreaticobiliary secretion-diversion guide (e.g., as shown in FIGS. 1-4), in accordance with some applications of the present invention. For some applications, an anchoring system 140 is used to fix a pancreaticobiliary secretion-diversion guide in place within the gastrointestinal tract of a subject. Typically, anchoring system 140 comprises one or more helical anchors 80 and anchoring mount 100. Anchor 80 is generally the same as anchor 80 described hereinabove. FIG. 5A shows anchor 80 deployed within the first section of duodenum 10, duodenal bulb 19. Anchor 80 pushes against the walls of duodenal bulb 19 and applies pressure to the wall of duodenal bulb 19 in order to fix a pancreaticobiliary secretion-diversion guide in place. Anchor 80 typically comprises retrieval element 82 described hereinabove with reference to FIG. 2A-B.

For some applications, anchor 80 is coupled to an anchoring mount 100. Anchoring mount 100 is typically deployed within the gastrointestinal tract, in a location that is downstream of helical anchor 80. Anchoring mount 100 typically comprises two or more, e.g., four, longitudinal struts 32, configured to be aligned in parallel with a longitudinal axis of the gastrointestinal tract. Longitudinal struts 32 typically contact the walls of the intestine in order to apply pressure to the walls and maintain a pancreaticobiliary secretion-diversion guide in place within the gastrointestinal tract of the subject.

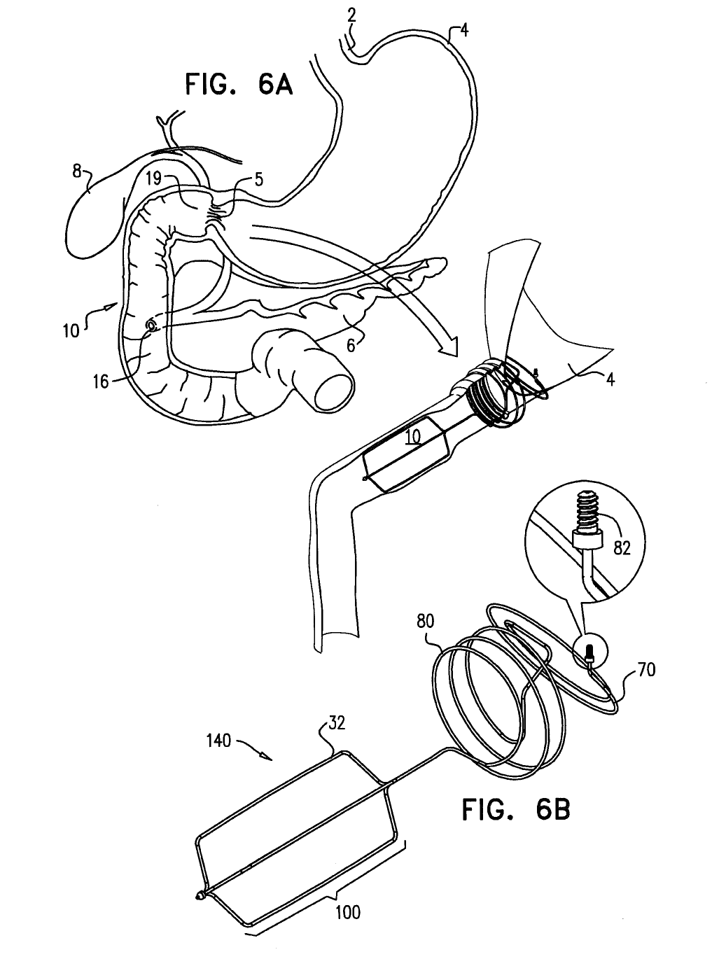

Reference is made to FIGS. 6A-B, which are schematic illustrations of an alternative configuration of the anchoring system 140 for use with a pancreaticobiliary secretion-diversion guide (e.g., as shown in FIGS. 1-4), in accordance with an application of the present invention. For some applications anchoring system 140 further comprises an additional intragastric anchor 70. Intragastric anchor 70 is typically the same as intragastric anchor 70 described hereinabove.

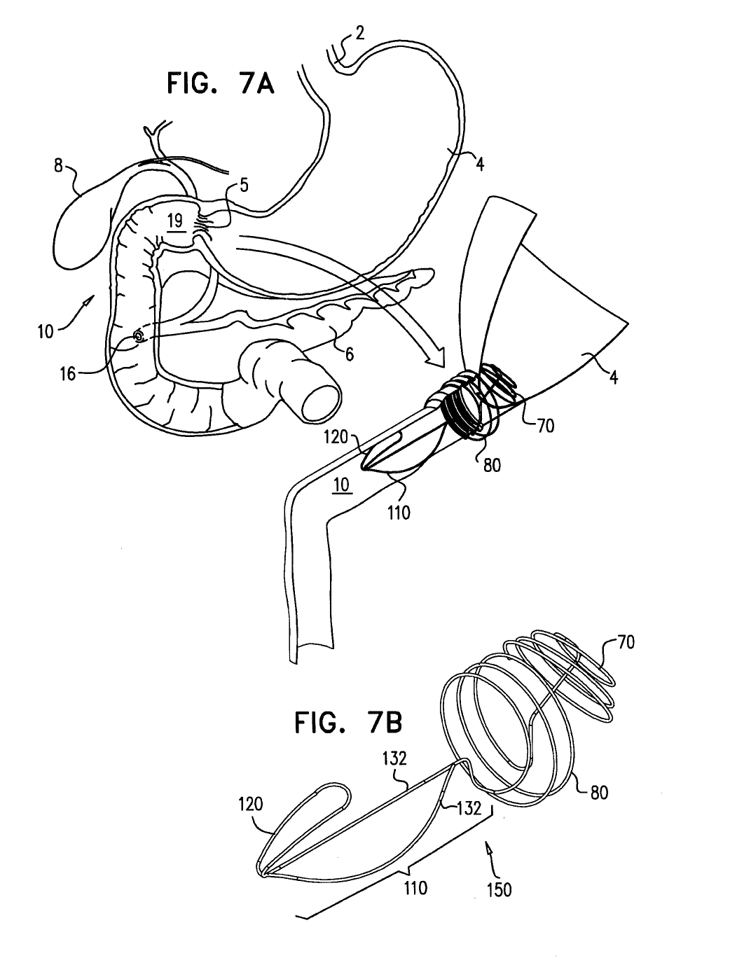

Reference is made to FIGS. 7A-B, which are schematic illustrations of anchoring system 150 for use with a pancreaticobiliary secretion-diversion guide (e.g., as shown in FIGS. 1-4), in accordance with some applications of the present invention. For some applications, anchoring system 150 is used to fix a pancreaticobiliary secretion-diversion guide in place within the gastrointestinal tract of a subject. Typically, anchoring system 150 comprises one or more helical anchors 80 and anchoring mount 110. Anchor 80 is generally the same as anchor 80 described hereinabove. FIG. 7A shows anchor 80 deployed within the first section of duodenum 10, duodenal bulb 19. Anchor 80 pushes against the walls of duodenal bulb 19 and applies pressure to the wall of duodenal bulb 19 in order to fix the pancreaticobiliary secretion-diversion guide in place.

For some applications, anchor 80 is coupled to an anchoring mount 110. Anchoring mount 110 is typically deployed within the gastrointestinal tract, in a location that is downstream of helical anchor 80. Anchoring mount 110 typically comprises two or more longitudinal struts 132. For some applications, at least one of struts 132 is configured to be aligned in parallel with a longitudinal axis of the gastrointestinal tract. For some applications, at least one of struts 132 is curved. FIG. 7A shows a curved strut 132 contacting the wall of the intestine in order to apply pressure to the wall and maintain a pancreaticobiliary secretion-diversion guide in place within the gastrointestinal tract of the subject.

Anchoring mount 110 typically comprises an additional flexible and elastic anchoring element 120. Element 120 typically contacts a wall of the intestine in order to apply pressure to the wall and maintain a pancreaticobiliary secretion-diversion guide in place within the gastrointestinal tract of the subject.

For some applications, anchoring system 150 further comprises an additional intragastric anchor 70. Intragastric anchor 70 is typically the same as intragastric anchor 70 described hereinabove. For some applications, anchors 70 and/or anchor 80 may comprise a retrieval element, e.g., similar to element 82 described hereinabove.

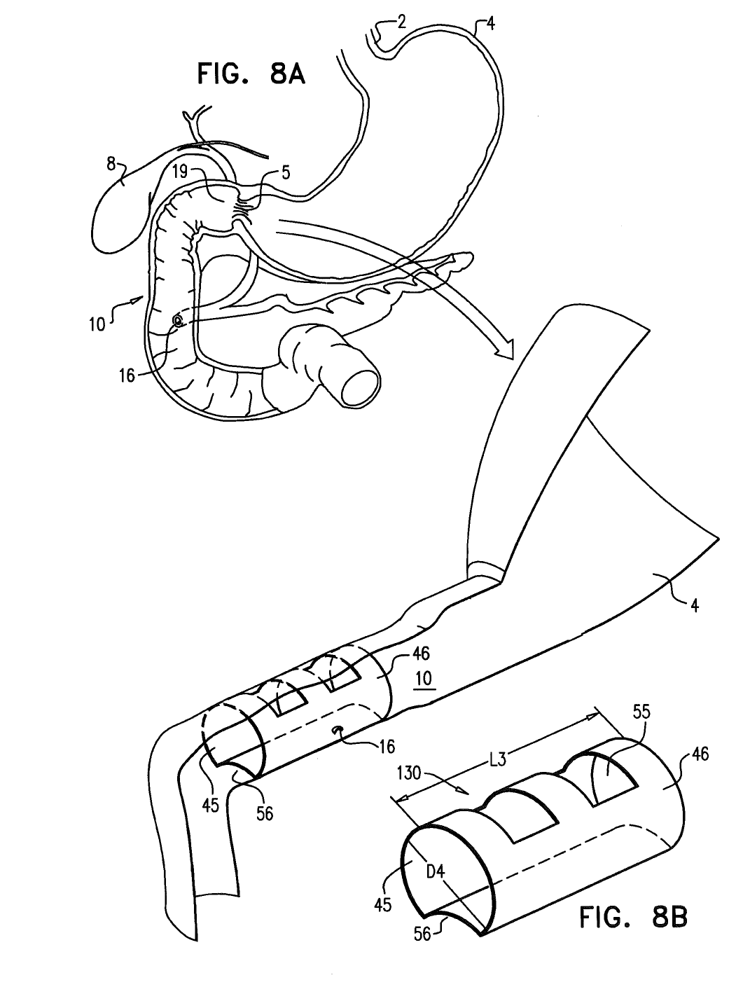

Reference is made to FIGS. 8A-B, which are schematic illustrations of a pancreaticobiliary secretion-diversion guide 130, in accordance with some applications of the present invention. For some applications, pancreaticobiliary secretion-diversion guide 130 is shaped to define at least a portion of a tube, the tube comprising a tube wall, the tube wall having an inner surface 45 and an outer surface 46, the tube wall inner surface defining a lumen for passage of chyme therethrough. Typically the lumen of guide 130 has a characteristic diameter D4 of 20-35 mm, e.g., 25-30 mm (it being understood that guide 130 as shown in FIGS. 8A-B does not form a complete circle). Guide 130 typically has a length L3 of at least 60 cm, or less than 110 cm or between 60 and 110 cm. When deployed within the gastrointestinal tract of the subject, guide 130 typically extends from a location in duodenum 10 to an area that is in the upper or in the mid jejunum. Alternatively, guide 130 is of sufficient length in order to extend from a location in duodenum 10 to an area that is in the lower jejunum or the ileum of the small intestine.

The tube wall of guide 130 typically comprises an aperture portion, shaped to define one or more apertures 55, e.g., 1-5 or 5-50 apertures. For some applications, the apertures have a cross-sectional area of at least 1 cm2, although apertures of smaller cross-sectional area (i.e., less than 1 cm2) are also suitable for use in some applications of the present invention. When deployed within the gastrointestinal tract, typically within duodenum 10, guide 130 contacts the wall of the gastrointestinal tract of the subject, and apertures 55 provide contact between chyme within the lumen and the wall of the gastrointestinal tract.

The tube wall is further shaped to define a channel 56, which is configured to collect pancreaticobiliary secretions from an anatomical entry location into the gastrointestinal tract, e.g., duodenal papilla 16 and to inhibit contact of the pancreaticobiliary secretions with the food within the lumen of the tube.

Guide 130 is configured to collect pancreaticobiliary secretions from an anatomical entry location into duodenum 10, e.g., duodenal papilla 16, and deliver the pancreaticobiliary secretions to a location in the gastrointestinal tract that is distal to the duodenal papilla. For some applications, guide 130 transfers the pancreaticobiliary secretions to a location that is beyond the duodenum, e.g., beyond the ligament of Treitz which is the final section of the duodenum. For some applications, guide 130 diverts the pancreaticobiliary secretions to a location that is at least 40 cm or less than 80 cm, or between 40 and 80 cm beyond the ligament of Treitz.

Guide 130 is typically deployed within the gastrointestinal tract of the subject, such that channel 56 faces duodenal papilla 16. Guide 130 is typically disposed within duodenum 10 in a location that is in the vicinity of duodenal papilla 16, such that secretions entering the duodenum at papilla 16 are directly collected into channel 56 of guide 130. The pancreaticobiliary secretions flow through distally in channel 56 of guide 130, and are typically discharged from the channel in an area that is in the upper or in the mid jejunum of the small intestine.

Channel 56 of guide 130 generally inhibits contact between the pancreaticobiliary secretions and chyme passing within the guide lumen, thereby reducing emulsification and formation of micelles of ingested fat, and disrupting the process of fat digestion in the body.

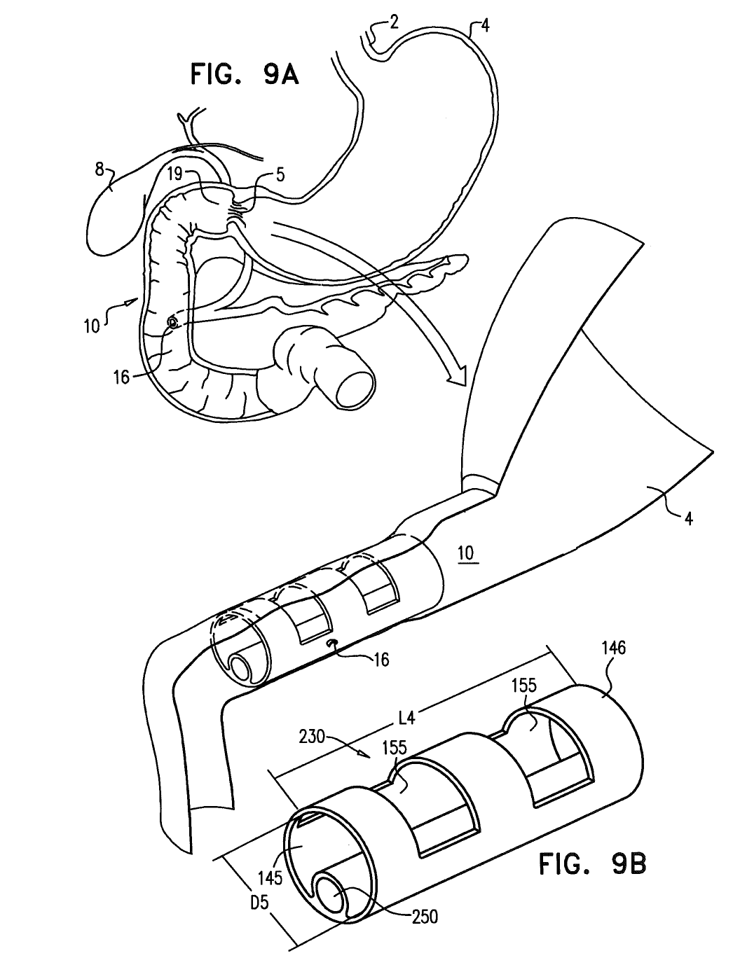

Reference is made to FIGS. 9A-B, which are schematic illustrations of a pancreaticobiliary secretion-diversion guide 230, in accordance with some applications of the present invention. For some applications, pancreaticobiliary secretion-diversion guide 230 is shaped to define at least a portion of a tube, the tube comprising a tube wall, the tube wall having an inner surface 145 and an outer surface 146, the tube wall inner surface defining a lumen for passage of chyme therethrough. Typically the lumen of guide 230 has a characteristic diameter D5 of 20-35 mm, e.g., 25-30 mm. Guide 230 typically has a length L4 of at least 60 cm, or less than 110 cm or between 60 and 110 cm. When deployed within the gastrointestinal tract of the subject, guide 230 typically extends from a location in duodenum 10 to an area that is in the upper or in the mid jejunum. Alternatively, guide 130 is of sufficient length in order to extend from a location in duodenum 10 to an area that is in the lower jejunum or the ileum of the small intestine.

The tube wall of guide 230 typically comprises an aperture portion, shaped to define one or more apertures 155, e.g., 1-5 or 5-50 apertures 155. When deployed within the gastrointestinal tract, typically within duodenum 10, guide 230 contacts the wall of the gastrointestinal tract of the subject, and apertures 155 provide contact between chyme within the lumen and the wall of the gastrointestinal tract. For some applications, the apertures have a cross-sectional area of at least 1 cm2, although apertures of smaller cross-sectional area (i.e., less than 1 cm2) also are suitable for use in some applications of the present invention. Typically, guide 230 makes contact with a complete circumference of the duodenum.

For some applications, inner surface 145 of the tube wall of guide 230 is coupled to an additional inner tube 250. Typically, tube 250 is shaped to define a tube lumen configured for passage of pancreaticobiliary secretions therethrough. Tube 250 is typically shaped to define an orifice in a lateral wall of the lumen (not shown) having a diameter of 5-20 mm, e.g., 10-20 mm, which is configured to collect pancreaticobiliary secretions from an anatomical entry location into the gastrointestinal tract, e.g., duodenal papilla 16, and to inhibit contact of the pancreaticobiliary secretions with the chyme within the lumen of the tube. For such applications, the orifice has a diameter that is sufficient in size to surround at least the entire diameter of the duodenal papilla.

For some applications, inner tube 250 has a length of at least 60 cm, or less than 110 cm, or between 60 and 110 cm. Inner tube 250 is configured to collect pancreaticobiliary secretions from an anatomical entry location into duodenum 10, e.g., duodenal papilla 16, and deliver the pancreaticobiliary secretions to a location in the gastrointestinal tract that is distal to the duodenal papilla. For some applications, guide 230 transfers the pancreaticobiliary secretions to a location that is beyond the duodenum, e.g., beyond the ligament of Treitz. For some applications, guide 230 diverts the pancreaticobiliary secretions to a location that is at least 40 or less than 80 cm, or between 40 and 80 cm beyond the ligament of Treitz.

Guide 230 is typically deployed within the gastrointestinal tract of the subject, such that the orifice of inner tube 250 faces duodenal papilla 16. Guide 230 is typically disposed within duodenum 10 in a location that is in the vicinity of duodenal papilla 16, such that secretions entering the duodenum at papilla 16 are directly collected into tube 250 of guide 230. The pancreaticobiliary secretions flow distally in tube 250 of guide 130, and are typically discharged from the tube in an area that is in the upper or in the mid jejunum. Alternatively, tube 250 is of sufficient length in order to deliver the pancreaticobiliary secretions to an area that is in the lower jejunum or the ileum of the small intestine.

Inner tube 250 of guide 230 generally inhibits contact between the pancreaticobiliary secretions and chyme passing within the guide lumen, thereby reducing emulsification and formation of micelles of ingested fat, and disrupting the process of fat digestion in the body.

For some applications, a pancreaticobiliary secretion diversion guide as described for example with reference to FIG. 1, or parts thereof, is used in combination with guide 230. For example, guide 30 (described with reference to FIG. 1) may replace inner tube 250 (configurations not shown).

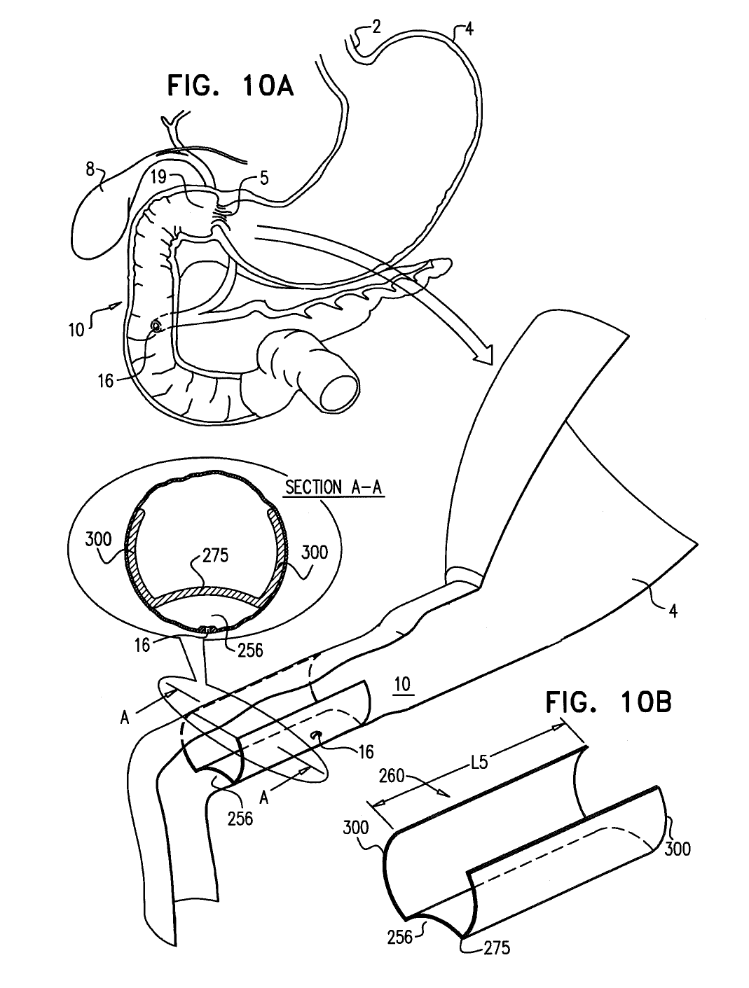

Reference is made to FIGS. 10A-B, which are schematic illustrations of a pancreaticobiliary secretion-diversion guide 260, in accordance with some applications of the present invention. For some applications, pancreaticobiliary secretion-diversion guide 260 comprises a guide body 275 having an inner surface and an outer surface. The outer surface of the guide body is typically shaped to define a channel 256, which is configured to collect pancreaticobiliary secretions from an anatomical entry location into the gastrointestinal tract, e.g., duodenal papilla 16 and to inhibit contact of the pancreaticobiliary secretions with the chyme within the duodenum. The chyme passing through duodenum 10 typically comes in contact with the inner surface of guide body 275.

Guide 260 is typically deployed within the gastrointestinal tract of the subject, such that channel 256 faces duodenal papilla 16. Guide 260 is typically disposed within duodenum 10 in a location that is in the vicinity of duodenal papilla 16, such that secretions entering the duodenum at papilla 16 are directly collected into channel 256 of guide 260. The pancreaticobiliary secretions flow through distally in channel 256 of guide 260, and are typically discharged from the channel in an area that is distal to the duodenal papilla. For some applications, the secretions are discharged in the upper or in the mid jejunum of the small intestine. For some applications, the pancreaticobiliary secretions are discharged in a location that is at least 40 cm or less than 80 cm, or between 40 and 80 cm beyond the ligament of Treitz.

Channel 256 and guide body 275 of guide 260 generally inhibit contact between the pancreaticobiliary secretions and chyme passing within the duodenum, thereby reducing emulsification and formation of micelles of ingested fat, and disrupting the process of fat digestion in the body.

Guide 260 typically has a length L5 of at least 60 cm, or less than 110 cm or between 60 and 110 cm. When deployed within the gastrointestinal tract of the subject, guide 230 typically extends from a location in duodenum 10 to an area that is in the upper or in the mid jejunum. Alternatively, guide 260 is of sufficient length in order to extend from a location in duodenum 10 to an area that is in the lower jejunum or the ileum of the small intestine.

Guide 260 comprises one or more arms 300, coupled to the guide body, and configured to apply pressure to the wall of the gastrointestinal tract in order to maintain the pancreaticobiliary secretion-diversion guide in place within the intestine.

Reference is made to FIG. 10A which includes a cross-sectional schematic illustration, indicated by A, of guide 260 when deployed within a gastrointestinal tract of the subject. Arms 300 are shown pressing against a section of the intestine wall and applying an outward force to the intestine in order to maintain guide 260 in place.

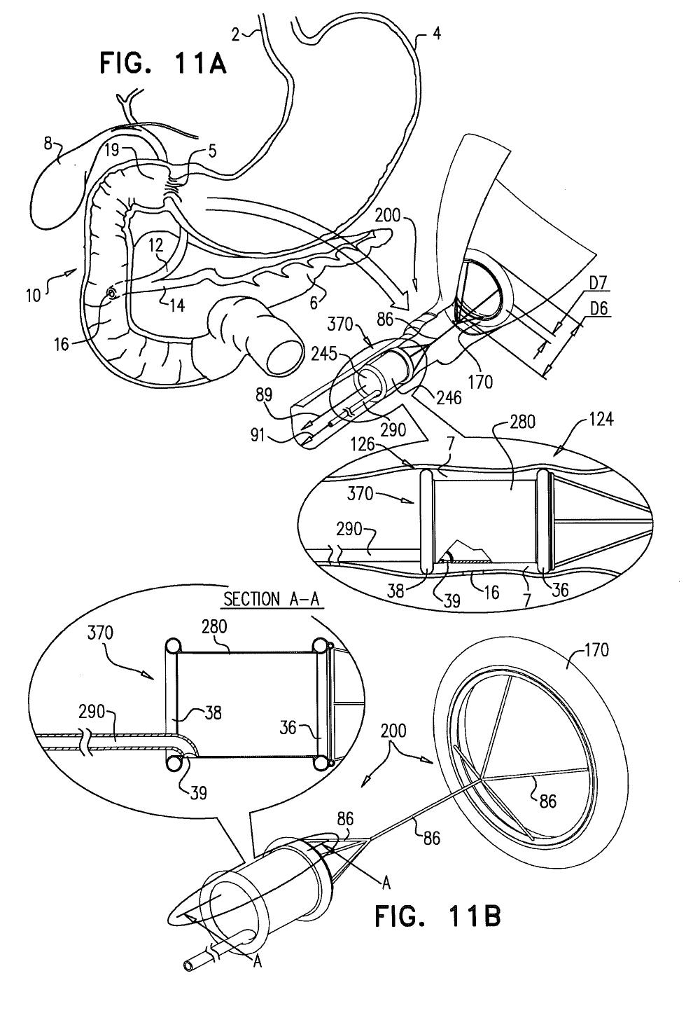

Reference is made to FIGS. 11A-B, which are schematic illustrations of apparatus 200 comprising a pancreaticobiliary secretion-diversion guide 370, in accordance with some applications of the present invention. For some applications, pancreaticobiliary secretion-diversion guide 370 comprises a proximal portion 124 and a distal portion 126 and first anchor 36 and second anchor 38 coupled to the proximal and distal portions respectively. First anchor 36 and second anchor 38 are each configured to contact and apply pressure to the wall of the gastrointestinal tract in order to maintain pancreaticobiliary secretion-diversion guide 370 in place within the gastrointestinal tract. For some applications, first and second anchors 36 and 38 are shaped to define ring anchors, each having a diameter of diameter of 20-35 mm when deployed within the gastrointestinal tract.

Pancreaticobiliary secretion-diversion guide 370 comprises a collection-facilitation tube 280, located between the first 36 and second 38 anchors. Collection-facilitation tube 280 is shaped to define inner surface 245 and outer surface 246. Inner surface 245 defines a lumen for passage of food therethrough (indicated by arrow 89), and outer surface 246 is disposed to inhibit contact of pancreaticobiliary secretions with food within the lumen. Outer surface 246 is additionally configured to define a space 7 between outer surface 246 and the gastrointestinal tract, for collecting pancreaticobiliary secretions. Collection-facilitation tube 280 typically has a diameter of at least 15 mm and/or less than 30 mm, e.g., at least 20 mm and/or less than 25 mm.

Collection-facilitation tube 280 is typically flexible. Pancreaticobiliary secretion-diversion guide 370 typically does not comprise a stent that surrounds collection-facilitation tube 280.

Pancreaticobiliary secretion-diversion guide 370 further comprises a diversion tube 290, configured to be in fluid communication with space 7 through aperture 39 in collection-facilitation tube 280. Diversion tube 290 passes pancreaticobiliary secretions from space 7 to a location in the gastrointestinal tract that is distal to the anatomical entry location, and inhibits contact of the pancreaticobiliary secretions with food that has passed through the lumen of tube 280. Diversion tube 290 typically has a diameter of 4-20 mm, e.g., 5-10 mm, such as 6-8 mm. Arrow 91 indicates the direction of flow of pancreaticobiliary secretions in diversion tube 290.

For some applications, secretion-diversion guide 370 transfers the pancreaticobiliary secretions to a location that is beyond the duodenum, e.g., beyond the ligament of Treitz, which is the final section of the duodenum. For some applications, secretion-diversion guide 370 diverts the pancreaticobiliary secretions to a location that is at least 40 cm or less than 80 cm or between 40 and 80 cm beyond the ligament of Treitz. For some applications, guide 370 is configured to divert the pancreaticobiliary secretions to any location within the small intestine that is distal to the ligament of Treitz (e.g., a location that is in the lower jejunum or the ileum). Accordingly, diversion tube 290 is configured (i.e., sufficient in length) to deliver the pancreaticobiliary secretions to the desired location within the small intestine. Typically, the length and other characteristics of diversion tube 290 are the same or generally similar to those described with reference to distal portion 28 shown in FIGS. 1A-C, except with respect to differences specifically noted herein.

For some applications, apparatus 200 further comprises an intragastric anchor 170, which is configured for deployment within the stomach of the subject (e.g., within the pyloric antrum of the stomach). Intragastric anchor 170 is coupled to guide 370 and assists in maintaining guide 370 in place within the gastrointestinal tract. Intragastric anchor 170 is typically configured to remain within the stomach of the subject and accordingly is sufficient in size, and shaped in a manner, that prevents it from passing through the pylorus. Optionally, portions of intragastric anchor 170 contact the wall of the stomach and may apply pressure to the wall of the stomach in order to maintain guide 370 in place.

For some applications, intragastric anchor 170 comprises a fluid-filled sheath, i.e., a gas or a liquid, e.g., saline. For some applications, intragastric anchor 170 is further coated with a biocompatible and biologically-inert material such as polytetrafluoroethylene (PTFE) and/or silicone. Intragastric anchor 170 is shown having a toroidal shape by way of illustration and not limitation. Intragastric anchor 170 may have any suitable shape that is configured to remain within the stomach to facilitate anchoring of guide 370 within the gastrointestinal tract of the subject. Typically, intragastric anchor 170 has a center-line diameter D6 of 14-100 mm, e.g., 15-35 mm, e.g., 18-25 mm, and a cross-sectional diameter D7 of a section thereof is typically 2-10 mm.

Typically, one or more flexible connecting elements 86, e.g., connecting wires, couple intragastric anchor 170 to first anchor 36 of guide 370. Connecting elements 86 typically comprise a flexible material such as nitinol or nylon. For some applications, connecting elements 86 may further comprise or be coated with a biocompatible and biologically-inert material such as polytetrafluoroethylene (PTFE) and/or silicone. For some applications, connecting elements 86 comprise one or more helically-shaped elements and/or elastic portions, allowing elements 86 to deform in response to peristaltic motion of the gastrointestinal tract.

It is noted that apparatus 200 may comprise any other intragastric anchor described herein, e.g., intragastric anchors 70, 171 or 172 and similarly, intragastric anchor 170 may be used with any apparatus for use with pancreaticobiliary secretions shown in FIGS. 1-16.

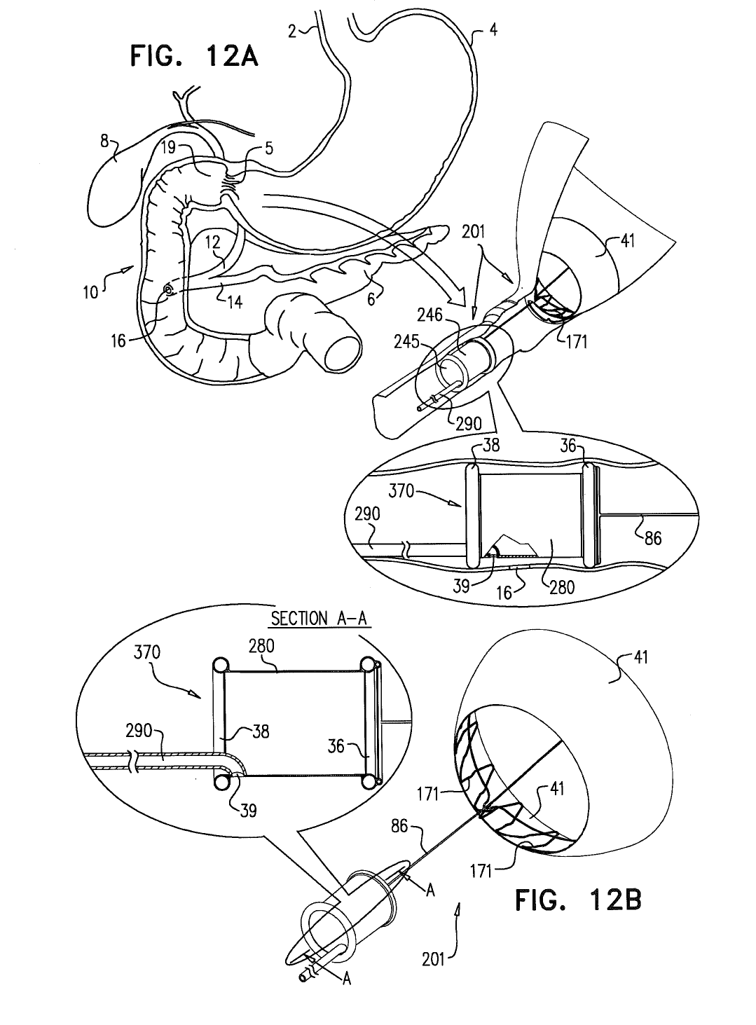

Reference is made to FIGS. 12A-B, which are schematic illustrations of apparatus 201, which comprises pancreaticobiliary secretion-diversion guide 370, coupled to intragastric anchor 171, in accordance with some applications of the present invention. Intragastric anchor 171 is coupled to guide 370 and assists in maintaining guide 370 in place within the gastrointestinal tract. Intragastric anchor 171 comprises a three-dimensional structure comprising a plurality of nitinol or stainless steel wires (or another material) which can be deployed and/or expanded within the stomach of the subject. As described hereinabove with reference to intragastric anchor 170, intragastric anchor 171 is configured to remain in the stomach and not pass through the pylorus. Optionally, portions of intragastric anchor 170 contact the wall of the stomach and may apply pressure to the wall of the stomach in order to maintain guide 370 in place. For some applications, intragastric anchor 171 is coated by a layer 41 of silicone and/or polytetrafluoroethylene (PTFE) as shown in FIG. 12A-B. Intragastric anchor 171 is typically coupled to guide 370 by one or more flexible connecting elements 86, as described hereinabove with reference to intragastric anchor 170. Guide 370 is as described hereinabove with reference to FIGS. 11A-B.

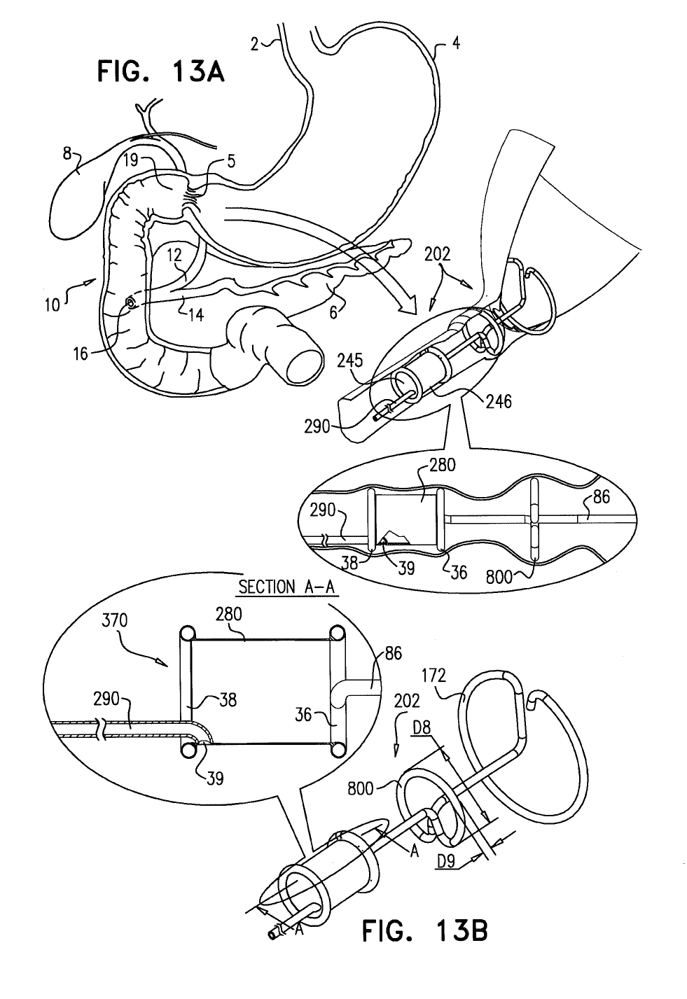

Reference is made to FIGS. 13A-B, which are schematic illustrations of apparatus 202, which comprises a pancreaticobiliary secretion-diversion guide 370, in accordance with some applications of the present invention. Guide 370 is as described hereinabove with reference to FIGS. 11A-B. For some applications, apparatus 202 further comprises a support anchor 800, which is disposed proximally to collection-facilitation tube 280 of guide 370. Typically (as shown in FIG. 13A), support anchor 800 is configured to be deployed within a duodenal bulb of the subject. Support anchor 800 typically has a center-line diameter D8 of 25-40 mm and a cross-sectional diameter D9 of a section thereof that is 2-6 mm, when not constrained by the gastrointestinal tract. Support anchor 800 typically assumes a center-line diameter D8 of 30-60 mm when deployed within the gastrointestinal tract. Support anchor 800 is configured to apply pressure to the wall of the gastrointestinal tract in order to maintain pancreaticobiliary secretion-diversion guide 370 in place within the gastrointestinal tract. For some applications, support anchor 800 comprises metal supports which apply outward pressure against the wall of the gastrointestinal tract, thus increasing anchoring of apparatus 202 within the gastrointestinal tract.

For some applications, support anchor 800 may be shaped as helix having two or more turns (configuration not shown).

For some applications, apparatus 202 further comprises intragastric anchor 172. Intragastric anchor 172 assists in maintaining guide 370 in place within the gastrointestinal tract and is typically configured to remain within the stomach of the subject and not pass through the pylorus. Optionally, portions of intragastric anchor 172 contact the wall of the stomach and may apply pressure to the wall of the stomach in order to maintain guide 370 in place. Intragastric anchor 172 typically but not necessarily comprises a shape memory alloy such as nitinol or stainless steel. For some applications, intragastric anchor 172 is coated with a biocompatible and biologically-inert material such as polytetrafluoroethylene (PTFE) and/or silicone.

For some applications, intragastric anchor 172 is surrounded by a silicone sheath inflated with a fluid, i.e., a gas or a liquid, e.g., saline. Intragastric anchor 172 is typically coupled to guide 370 by one or more flexible connecting elements 86, as described hereinabove with reference to intragastric anchor 170. It is also noted that apparatus 202 may comprise any other intragastric anchor described herein, e.g., intragastric anchors 70, 170 or 171 and similarly, intragastric anchor 172 may be used with apparatus 20, 200, 201 and/or any apparatus for use with pancreaticobiliary secretions shown in FIGS. 1-16.

It is further noted that for some applications, apparatus 200, 201, and/or 202 do not comprise an intragastric anchor.

Support anchor 800 is typically coupled to intragastric anchor 172 and to collection-facilitation tube 280 of guide 370 by one or more flexible connecting elements 86. Connecting elements 86 typically comprise a flexible material such as nitinol or nylon. For some applications, connecting elements 86 may further comprise or be coated with a biocompatible and biologically-inert material such as polytetrafluoroethylene (PTFE) and/or silicone. For some applications, connecting elements 86 comprise a helix and/or an elastic portion distal and/or proximal to support anchor 800, allowing elements 86 to deform in response to peristaltic motion of the gastrointestinal tract.

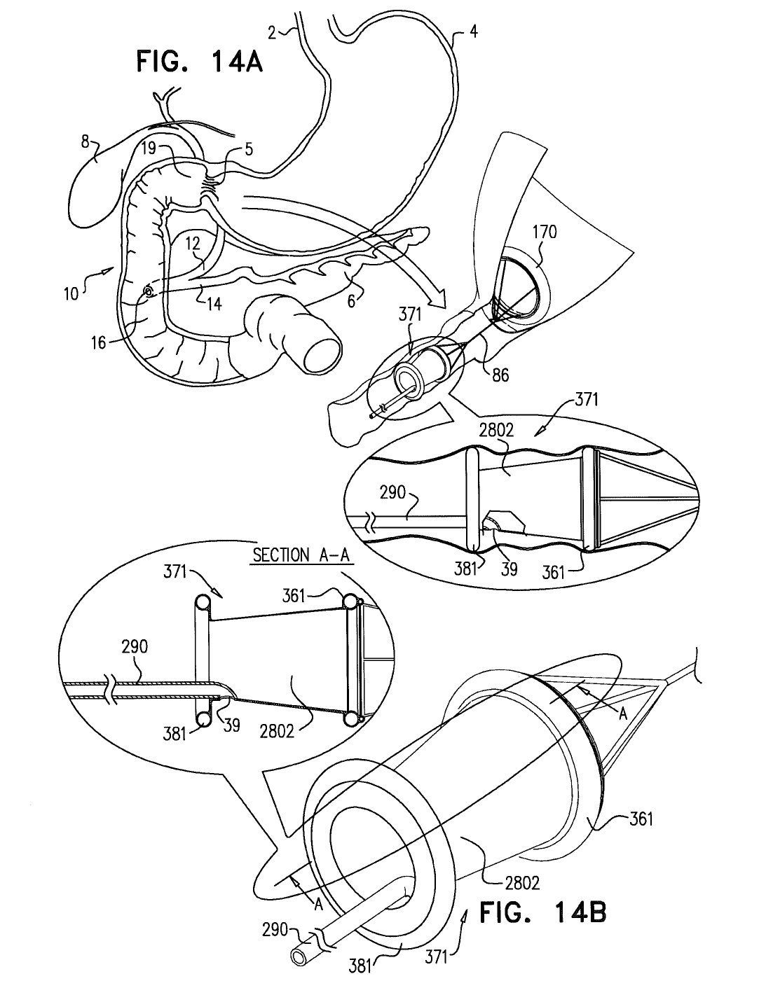

Reference is made to FIGS. 14A-B, which are schematic illustrations of pancreaticobiliary secretion-diversion guide 371, in accordance with an application of the present invention. Guide 371 is generally similar to guide 370 as described herein with reference to FIGS. 11A-B except where stated otherwise. Pancreaticobiliary secretion-diversion guide 371 typically comprises first anchor 361 and second anchor 381, which are configured to contact and apply pressure to the wall of the gastrointestinal tract, in order to maintain pancreaticobiliary secretion-diversion guide 371 in place within the gastrointestinal tract. Pancreaticobiliary secretion-diversion guide 371 comprises a frustoconical collection-facilitation tube 2802, located between the first 361 and second 381 anchors. The frustoconical shape of secretion-diversion guide 2802 typically facilitates collection of the pancreaticobiliary secretions by diversion tube 290 through aperture 39. Pancreaticobiliary secretion-diversion guide 371 is shown coupled to intragastric anchor 170 by way of illustration and not limitation. Guide 371 may be coupled to any intragastric anchor described herein. Alternatively, guide 371 is not coupled to an intragastric anchor.

Collection-facilitation tube 2802 is typically flexible. Pancreaticobiliary secretion-diversion guide 371 typically does not comprise a stent that surrounds collection-facilitation tube 2802.

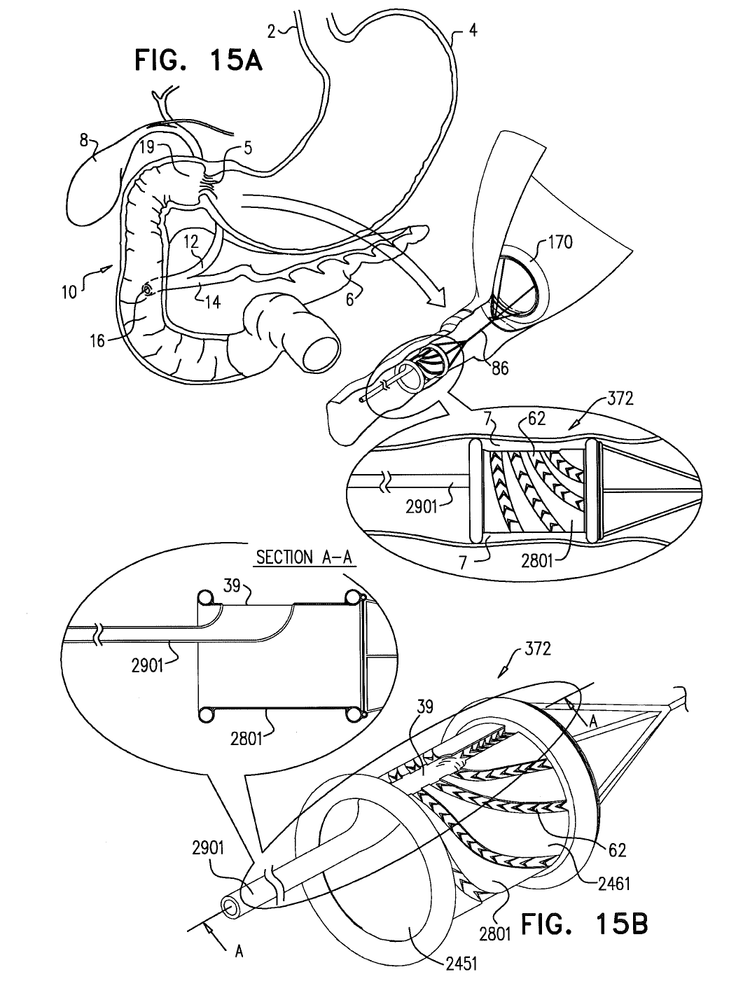

Reference is made to FIGS. 15A-B, which are schematic illustrations of pancreaticobiliary secretion-diversion guide 372, in accordance with an application of the present invention. Guide 372 is generally similar to guide 370 as described herein with reference to FIGS. 11A-B, except where stated otherwise. Pancreaticobiliary secretion-diversion guide 372 comprises a collection-facilitation tube 2801, which is shaped to define an inner surface 2451 and an outer surface 2461. Inner surface 2451 defines a lumen for passage of food therethrough, and outer surface 2461 is disposed to inhibit contact of pancreaticobiliary secretions with food within the lumen. Outer surface 2461 is additionally configured to define a space 7 between outer surface 2461 and the gastrointestinal tract for collecting pancreaticobiliary secretions. Outer surface 2461 of collection-facilitation tube 2801 is typically shaped to define a set of one or more grooves 62 configured to direct the pancreaticobiliary secretions into a diversion tube 2901 and facilitate collection of the pancreaticobiliary secretions by diversion tube 2901. For some applications, each of one or more grooves 62 has a depth of 0.5-3 mm. For some applications, as shown in FIGS. 15A-B, a proximal portion of diversion tube 2901 has a cross section that is larger than the cross section of a distal portion of diversion tube 2901, in order to facilitate collection of the pancreaticobiliary secretions into tube 2901. Alternatively, diversion tube 2901 has the same diameter along an entire length thereof, and a funnel is coupled to aperture 39 in order to facilitate collection of pancreaticobiliary secretions into tube 2901.

Collection-facilitation tube 2801 is typically flexible. Pancreaticobiliary secretion-diversion guide 372 typically does not comprise a stent that surrounds collection-facilitation tube 2801.

Pancreaticobiliary secretion-diversion guide 372 is shown coupled to intragastric anchor 170 by way of illustration and not limitation. Guide 371 may be coupled to any intragastric anchor described herein. Alternatively, guide 372 is not coupled to an intragastric anchor.

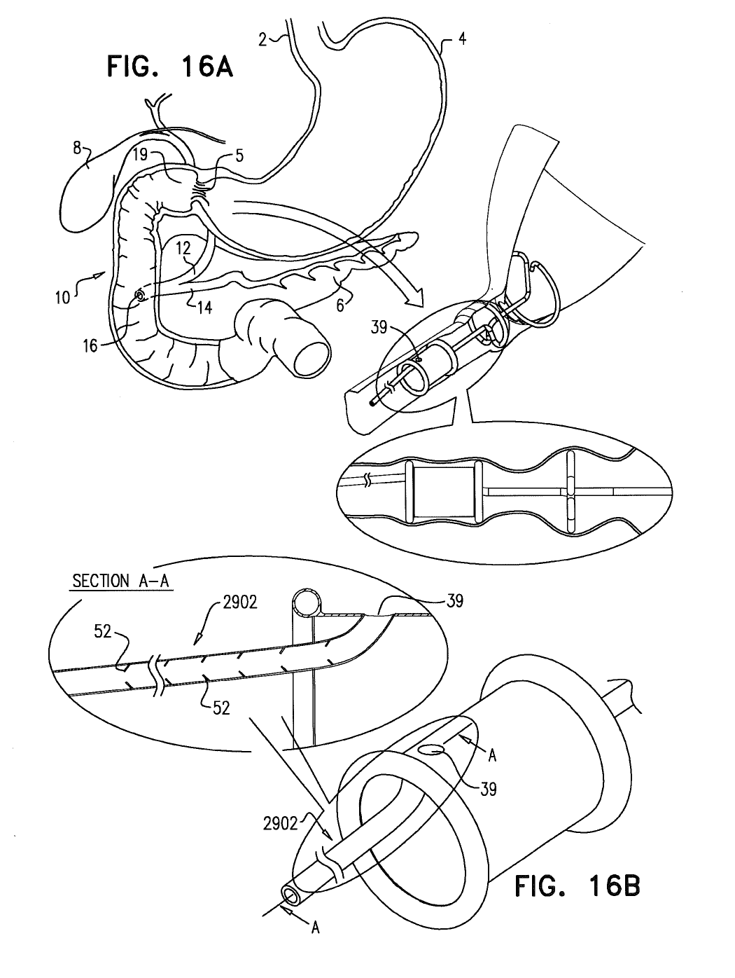

Reference is made to FIGS. 16A-B, which are schematic illustrations of a diversion tube 2902, in accordance with an application of the present invention. For some applications, diversion tube 2902 comprises a plurality of unidirectional valves 52 allowing the flow of pancreaticobiliary secretions in a distal direction along tube 2902. Unidirectional valves 52 are typically spaced 5-10 mm apart in tube 2902. Typically, the length and other characteristics of diversion tube 2902 are the same or generally similar to those described with reference to diversion tube 290 shown in FIGS. 11A-B, except with respect to differences specifically noted herein. Unidirectional valves 52 are typically configured to inhibit (even if not to completely eliminate) proximal flow of pancreaticobiliary secretions.

Diversion tube 2902 is shown in FIGS. 16A-B as being used with apparatus similar to apparatus 202 by way of illustration and not limitation. Diversion tube 2902 may be used, as appropriate, with any apparatus for use with pancreaticobiliary secretions described herein.

Reference is made to FIGS. 11A-16B. For some applications, the apparatus for use with pancreaticobiliary secretions which is described with reference to FIGS. 11A-16B further comprise a retrieval element (e.g., such as retrieval element 82 described herein with reference to FIG. 1).

Reference is still made to FIGS. 11A-16B. For some applications, as shown for example in FIGS. 11A-B, aperture 39 has a length that is 10-20% (e.g., 15%) of a length of collection-facilitation tube 280. Alternatively, aperture 39 has a length that is 20-50% of a length of the collection-facilitation tube, e.g., 30-50% of tube 2801, as shown for example in FIGS. 15A-B.

Reference is made to FIGS. 1A-16B. The apparatus for use with pancreaticobiliary secretions which is described herein may be surrounded by a facilitating sleeve during implantation. For some applications, a sleeve (not shown) surrounds all or some of the components of the apparatus, e.g., the anchoring system and/or the pancreaticobiliary secretion-diversion guide. Typically, the sleeve facilitates insertion of the apparatus for use with pancreaticobiliary secretions into a gastrointestinal tract of a subject.

Reference is still made to FIGS. 1A-16B. Components of the apparatus described herein, e.g., the anchoring system and/or the pancreaticobiliary secretion-diversion guide, typically but not necessarily comprise a shape memory alloy such as nitinol or stainless steel. For some applications, components of the apparatus described herein may comprise or be coated with a biocompatible and biologically-inert material such as polytetrafluoroethylene (PTFE) and/or silicone. For example, components of the anchoring system may be further surrounded by a silicone sheath which is configured to apply pressure to the wall of the gastrointestinal tract in order to maintain the pancreaticobiliary secretion-diversion guide in place. For some applications, the silicone sheath is inflated with a fluid, i.e., a gas or a liquid.

Typically, the apparatus described herein generally comprises a material with low permeability. For some applications, the apparatus material or coating provides a generally low coefficient of friction, e.g., less than 0.3.

For some applications, the apparatus described herein may be used as a drug delivery tool. The apparatus may comprise in its body, or be coated with, a substance, such as but not limited to, a medication (e.g., an antibiotic and/or an anti-inflammatory medication), a hormone, a bile acid resin, and/or another binder.

For some applications, one or more components of the apparatus described herein, e.g., the anchoring system and/or the pancreaticobiliary secretion-diversion guide, may comprise a biodegradable material, e.g., a biodegradable polymer, which gradually degrades, allowing the apparatus to leave the body.

For some applications, the apparatus described herein is configured to prevent tissue growth on an exterior surface of the apparatus (e.g., using a chemical coating on the exterior surface).

For some applications, a marker may be coupled to any component of the apparatus described herein, in order to enable detection by fluoroscopic imaging of the position and orientation of the apparatus within the gastrointestinal tract.

Reference is still made to FIGS. 1A-16B. Components of the apparatus described herein, e.g., the anchoring system and/or the pancreaticobiliary secretion-diversion guide, are configured for endoscopic deployment and retrieval and are typically inserted into the digestive tract of a subject using minimally invasive techniques such as endoscopy and/or endoscopic overtubing. Alternatively, components of the apparatus described herein, e.g., the anchoring system and/or the pancreaticobiliary secretion-diversion guide, may be deployed within the digestive tract by means of invasive surgery.

For some applications, the apparatus described herein may comprise fiber optics, biopsy tools, optical devices (e.g., a CCD camera) and/or other imaging devices.

Reference is still made to FIGS. 1A-16B. For some applications, the pancreaticobiliary secretion-diversion guide affects hormonal secretion and/or action. Many hormones and enzymes participate in the digestion process, some of which are released by the gastrointestinal tract, for example, (a) GIP (Glucose-dependent insulinotropic peptide), a digestive hormone which is synthesized by intestinal K cells, and (b) GLP-1 (Glucagon-like peptide-1), which is synthesized by intestinal L cells. These hormones typically play a role in the regulation of the digestive process, and may affect fat metabolism and insulin secretion and action. Some applications of the present invention may modify the secretion and/or the action of these hormones and consequently modify the digestive process (including but not limited to fat metabolism and insulin secretion and action).

Additionally or alternatively, the apparatus described herein or components thereof may trigger a sense of satiety in a subject. Typically, mechanoreceptors that are present in the stomach and proximal small intestine are sensitive to mechanical changes in these areas (e.g., pressure and/or stretching) that are usually caused by food passing through the gastrointestinal tract. These mechanical changes trigger a signal which leads to a sensation of satiety in the subject and as a result affects the appetite of the subject. For some applications, the apparatus described herein, or components thereof, are configured to contact and/or stretch and/or apply mechanical pressure to a portion of the gastrointestinal tract. Thus, the apparatus described herein may activate mechanoreceptors in the gastrointestinal tract leading to a sensation of satiety and, as a result, a reduction of food intake by the subject.

Accordingly, the pancreaticobiliary secretion-diversion guide as described herein is typically used for treatment of obesity, type II diabetes and other disease such as non-alcoholic fatty liver disease and/or non-alcoholic steatohepatitis.

Reference is made to the anchoring systems described with reference to FIGS. 1A-16B. For some applications, the anchoring systems described herein may be used for other implantable devices, including but not limited to, a bariatric sleeve. For some applications, the anchoring systems may be implanted for purposes of dilating areas in the gastrointestinal tract, e.g., in a patient with a constricted gastrointestinal tract due to cancer.

Additionally or alternatively, the anchoring systems described herein may be implanted for drug delivery purposes in any suitable location within the gastrointestinal tract. For example, the anchoring system may be implanted in the terminal ileum or any other region of the small or large intestine. For example, the anchors may release a drug for localized treatment of, e.g., neoplasia or an intestinal inflammatory disease, e.g., Crohn's disease or ulcerative colitis.

Reference is still made to the anchoring systems described with reference to FIGS. 1A-16B. For some applications, the anchoring system additionally or alternatively comprises tissue-penetrating anchors which penetrate tissue of the gastrointestinal tract in order to enhance anchoring of the secretion-diversion guide in the gastrointestinal tract. Typically the tissue-penetrating anchors are coupled to components of the anchoring system and/or to components of the secretion-diversion guide. The tissue-penetrating anchors are typically shaped to define any suitable shape, e.g., barbs, hooks, and/or spikes.

Although techniques of the present invention have generally been described herein as being applied to the stomach and small intestine, these techniques may additionally be used, mutatis mutandis, to treat other lumens of the gastrointestinal tract of a subject, such as the esophagus, or the large intestine.

It will be appreciated by persons skilled in the art that the present invention is not limited to what has been particularly shown and described hereinabove. Rather, the scope of the present invention includes both combinations and subcombinations of the various features described hereinabove, as well as variations and modifications thereof that are not in the prior art, which would occur to persons skilled in the art upon reading the foregoing description.

1. Apparatus for use with pancreaticobiliary secretions that enter a gastrointestinal tract of a subject at an anatomical entry location, the apparatus comprising:
a pancreaticobiliary secretion-diversion guide, comprising:
a proximal portion;
a distal portion;
first and second anchors coupled to the proximal and distal portions respectively and configured to apply pressure to the wall of the gastrointestinal tract in order to maintain the pancreaticobiliary secretion-diversion guide in place within the gastrointestinal tract;
a collection-facilitation tube, between the first and second anchors, having an inner surface and an outer surface, the inner surface defining a lumen for passage of food therethrough, the outer surface disposed to inhibit contact of the secretions with food within the lumen and configured to define a space between the outer surface and the gastrointestinal tract for collecting the secretions;
a diversion tube, configured to:
be in fluid communication with the space between the outer surface and the gastrointestinal tract,
pass pancreaticobiliary secretions from the space to a location in the gastrointestinal tract that is distal to the anatomical entry location, and
inhibit contact of the pancreaticobiliary secretions with food that has passed through the lumen; and
an intragastric anchor coupled to the pancreaticobiliary secretion-diversion guide, and configured to be located in a stomach of the subject and to maintain the pancreaticobiliary secretion-diversion guide in place.

2. The apparatus according to claim 1, wherein the outer surface of the collection-facilitation tube is shaped to define one or more grooves configured to facilitate collection of the pancreaticobiliary secretions into the diversion tube.

3. The apparatus according to claim 1, wherein the diversion tube comprises one or more unidirectional valves configured to direct the flow of pancreaticobiliary secretions in a distal direction in the diversion tube.

4. The apparatus according to claim 1, wherein the collection-facilitation tube is shaped to define a frustoconical collection-facilitation tube.

5. The apparatus according to claim 1, wherein the anchors comprise ring anchors.

6. The apparatus according to claim 5, wherein each ring anchor has a diameter of 20-35 mm.

7. The apparatus according to claim 1, wherein the intragastric anchor is toroidal and has a center line diameter of 15-100 mm.

8. The apparatus according to claim 7, wherein the intragastric anchor has a center line diameter of 15-35 mm.

9. The apparatus according to claim 8, wherein the intragastric anchor has a center line diameter of 18-25 mm.

10. The apparatus according to claim 1, wherein the intragastric anchor is toroidal and has a cross-sectional diameter of 2-10 mm.

11. The apparatus according to claim 1, further comprising one or more flexible connecting elements which couple the intragastric anchor to the pancreaticobiliary secretion-diversion guide.

12. The apparatus according to claim 11, wherein the one or more flexible connecting elements couple the intragastric anchor to the first anchor.

13. The apparatus according to claim 11, wherein the flexible connecting elements are configured to deform in response to peristaltic motion of the gastrointestinal tract.

14. The apparatus according to claim 11, wherein the flexible connecting elements comprise one or more helically-shaped elements.

15. The apparatus according to claim 1, wherein the diversion tube has a diameter of 4-20 mm.

16. The apparatus according to claim 1, wherein the collection-facilitation tube has a diameter of 20-25 mm.

17-25. (canceled)

26. The apparatus according to claim 1, wherein a proximal portion of the diversion tube has a cross section that is larger than a cross section of a distal portion of the diversion tube.

27. The apparatus according to claim 1, wherein the pancreaticobiliary secretion-diversion guide does not comprise a stent that surrounds the collection-facilitation tube.
